(19) United States
(12) Patent Application Publication
(10) Pub. No.: US 2014/0100354 A1
(43) Pub. Date: Apr. 10, 2014

(54) PEPTOID COMPOSITIONS AND METHODS OF USING THE SAME

(71) Applicants: Kent KIRSHENBAUM, New York, NY (US); Galia Maayan, New York, NY (US); Michael Ward, New York, NY (US)

(72) Inventors: Kent KIRSHENBAUM, New York, NY (US); Galia Maayan, New York, NY (US); Michael Ward, New York, NY (US)

(73) Assignee: NEW YORK UNIVERSITY

(21) Appl. No.: 13/644,369

(22) Filed: Oct. 4, 2012

(51) Int. Cl. C07K 7/64; C07D 301/03; C07K 5/097; C07B 41/06; C07K 7/06; C07K 5/117

(52) U.S. Cl. CPC C07K 7/64; C07K 7/06; C07K 5/1024; C07K 5/0821; C07B 41/06; C07D 301/03
USPC 530/321; 530/329; 530/330; 530/331; 530/328; 530/323; 568/322; 549/523; 560/255; 560/254

(57) ABSTRACT

Novel peptoids are disclosed that have a formula represented by the following formulae Ia and Ib:

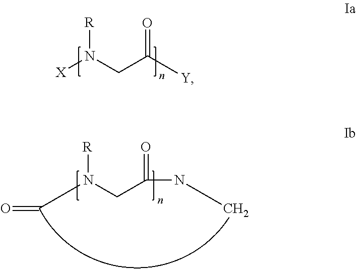

wherein X, Y, R, and n are as described herein. The peptoids demonstrate catalytic activity and are useful in substrate-selective catalytic transformations, including asymmetric catalytic transformations.

### RELATED APPLICATIONS

The present application claims the benefit under 35 U.S.C. §119 of U.S. Provisional Application Ser. No. 61/053,958 filed May 16, 2008. The contents of said provisional application is hereby incorporated by reference in its entirety.

### GOVERNMENT RIGHTS

This invention was made with government support under Grant No. 0645361 awarded by the NSF. Accordingly, the United States Government has certain rights in the invention.

### FIELD OF THE INVENTION

This invention relates to novel compositions containing acyclic and cyclic peptoids, and particularly, to the preparation and use of such compositions and corresponding peptoids as catalysts in various chemical reactions, such as the synthesis of enantiomerically pure organic compounds, and in various substrate-selective organic transformations, such as the asymmetric catalytic resolution of aromatic secondary alcohols.

### BACKGROUND OF THE INVENTION

The ability to mimic the structure and function of enzymes is a great challenge in bioorganic chemistry. Efforts have been made to mimic the structure of enzyme active sites as well as enzymatic activity and substrate selectivity. Since enzymes are actually proteins with complex folds that contain functional sites, such as recognition and catalytic sites, one way of mimicking an enzyme will be to generate an oligomeric backbone that contains key chemical functionalities as pendant groups displayed in a precise spatial relationship.

N-substituted glycine oligomers, or “peptoids”, are a family of peptidomimetic foldamers capable of adopting stable secondary structures. By employing a solid-phase synthesis protocol, a wide variety of side chains can be incorporated into peptoid sequences. Thus, the peptoid scaffold can be used as an efficient platform for different catalytic and recognition sites displayed in a specific manner, allowing the mimicry of enzymatic modes of action that promote catalytic function. Recent advances in the study of peptoids have allowed us to (1) develop techniques for controlling secondary structure and the presentation of side-chains and (2) incorporate chemical functionalities that may be suitable to provide catalytic centers, such as amino groups, carboxylic acids, imidazoles, alcohols, thiols, liganded metal ions, and stable free-radical nitroxides. These advances have enabled the construction of peptoid architectures which embed these groups in a highly controlled environment capable of discriminating potential reaction substrates.

### SUMMARY OF THE INVENTION

As set forth earlier herein, the present invention comprises novel N-substituted glycine cyclic and acyclic peptoid compositions and uses thereof. The peptoids may be useful in catalytic transformations. More particularly, the peptoids may be useful in substrate-selective catalysis and asymmetric catalytic resolution. These peptoids can accordingly include natural/normatural amino acids: beta-amino acids, D-amino acids and/or other proteinogenic and abiotic amino acids.

More particularly, the present invention relates to acyclic and cyclic peptoids having catalytic properties, according to formulae Ia or Ib:

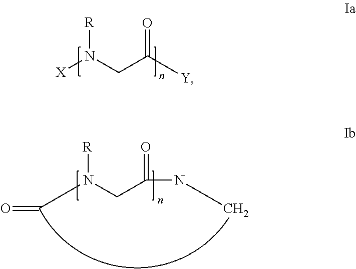

comprised of monomers according to formula II and formula III:

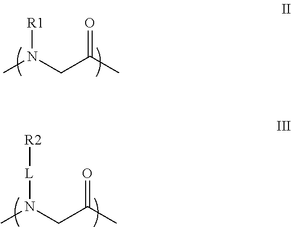

wherein

each R is independently substituted or unsubstituted alkyl, substituted or unsubstituted alkenyl, substituted or unsubstituted alkynyl, substituted or unsubstituted heterocycloalkyl, substituted or unsubstituted aryl, or substituted or unsubstituted heteroaryl;

each R¹ is independently substituted or unsubstituted alkyl, substituted or unsubstituted alkenyl, substituted or unsubstituted alkynyl, substituted or unsubstituted aryl or substituted or unsubstituted heteroaryl;

each R² is a group or substituent capable of participating in the catalysis of a chemical transformation;

L is a single bond, C₁-C₄ alkylene, —C₂-C₄ alkylene-O—, or —C₂-C₄ alkylene-O—C₁-C₄ alkylene-;

X is H, substituted or unsubstituted acyl; Y is NH₂, OH, acylamino, or acyloxy;

and n is an integer between 2-200;

or a salt thereof; and stereoisomers, isotopic variants and tautomers thereof;

provided that:
i) at least one of the monomers is of formula III.;
ii) each R¹ in the peptoid oligomer may be the same or different;
iii) each -L-R² in the peptoid oligomer may be the same or different; and
iv) the peptoid oligomer is other than:

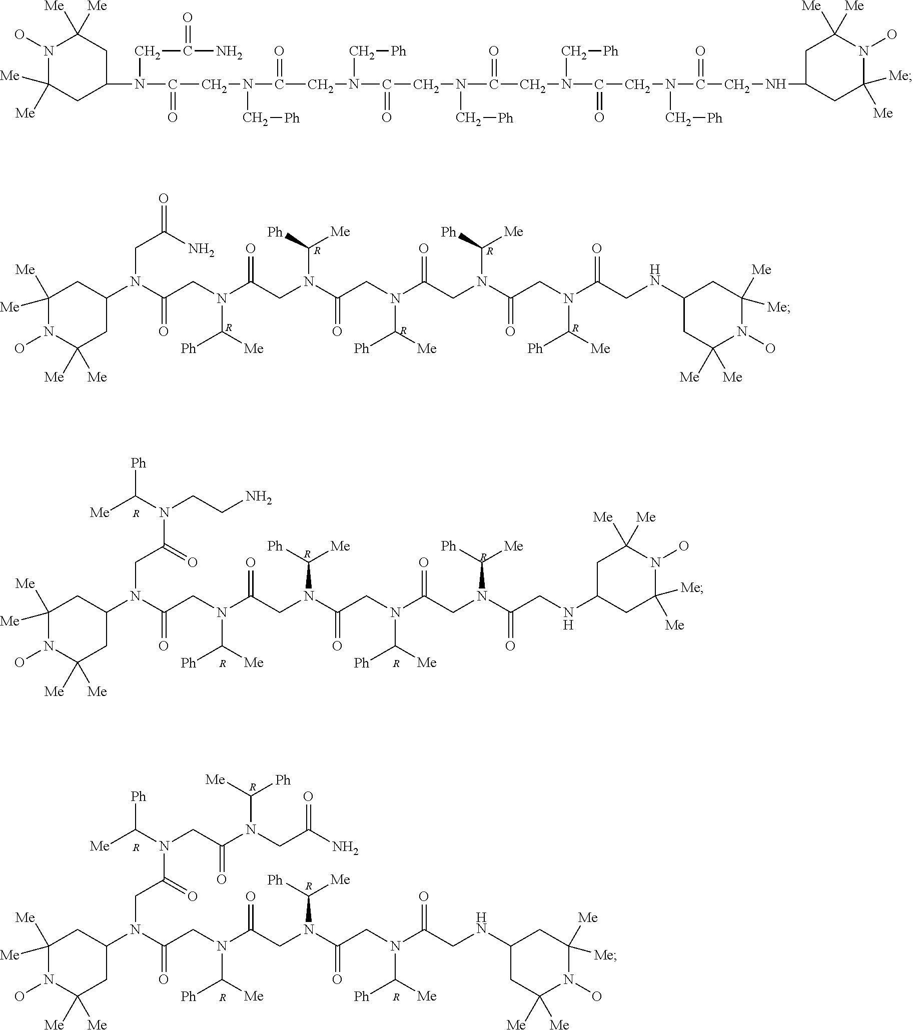

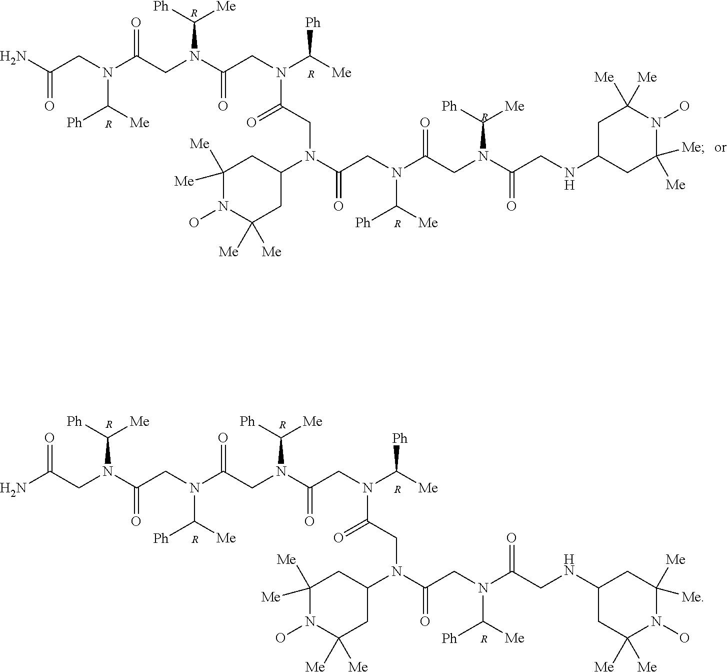

In one embodiment, the invention relates to a peptoid oligomer according to formula Ia or Ib, wherein 10-60% of the monomers are of formula III at the same time. In another embodiment, 10-20% of the monomers are of formula III at the same time.

In a further aspect, the present invention includes the use of the peptoids in chemical transformation.

In a further aspect, the present invention includes the use of the peptoids in substrate-selective catalytic transformation.

In a further aspect, the present invention includes the use of the peptoids in asymmetrical catalytic transformation.

In a further aspect, the present invention includes the use of the peptoids in asymmetrical catalytic resolution.

In additional aspects, this invention provides methods for synthesizing the peptoids of the invention, with representative synthetic protocols and pathways disclosed later on herein.

Other objects and advantages will become apparent to those skilled in the art from a consideration of the ensuing detailed description.

### DETAILED DESCRIPTION OF THE INVENTION

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that any of the moieties defined forth below may be substituted with a variety of substituents, and that the respective definitions are intended to include such substituted moieties within their scope. By way of non-limiting example, such substituents may include e.g. halo (such as fluoro, chloro, bromo), —CN, —CF₃, —OH, —OCF₃, O—CHF₂, C₁-C₆ alkyl, C₂-C₆ alkenyl, C₃-C₆ alkynyl, C₁-C₆ alkoxy, aryl and di-C₁-C₆ alkylamino. It should be further understood that the terms “groups” and “radicals” can be considered interchangeable when used herein.

The articles “a” and “an” may be used herein to refer to one or to more than one (i.e. at least one) of the grammatical objects of the article. By way of example “an analogue” means one analogue or more than one analogue.

‘Acyl’ or ‘alkanoyl’ refers to a radical —C(O)R²⁰, where R²⁰ is hydrogen, alkyl, cycloalkyl, cycloheteroalkyl, aryl, arylalkyl, heteroalkyl, heteroaryl, heteroarylalkyl as defined herein. Representative examples include, but are not limited to, formyl, acetyl, cylcohexylcarbonyl, cyclohexylmethylcarbonyl, benzoyl, benzylcarbonyl and the like.

‘Acylamino’ refers to a radical —NR²¹C(O)R²², where R²¹ is hydrogen, alkyl, cycloalkyl, cycloheteroalkyl, aryl, arylalkyl, heteroalkyl, heteroaryl or heteroarylalkyl and R²² is hydrogen, alkyl, alkoxy, cycloalkyl, cycloheteroalkyl, aryl, arylalkyl, heteroalkyl, heteroaryl or heteroarylalkyl, as defined herein. Representative examples include, but are not limited to, formylamino, acetylamino, cyclohexylcarbonylamino, cyclohexylmethyl-carbonylamino, benzoylamino and benzylcarbonylamino. In a particular embodiment, ‘acylamino’ refers to a group —NR^{B}′C(O)R^{A}′ wherein each R^{A}′ is independently selected from C₁-C₈ alkyl, —(CH₂)ₜ(C₆-C₁₀ aryl), —(CH₂)ₜ(C₅-C₁₀ heteroaryl), —(CH₂)ₜ(C₃-C₁₀ cycloalkyl), and —(CH₂)₁(C₅-C₁₁ heterocycloalkyl), wherein t is an integer from 0 to 4 and any aryl, heteroaryl, cycloalkyl or heterocycloalkyl groups present, may themselves be substituted by C₁-C₄ alkyl, halo, C₁-C₄ alkoxy, C₁₋₄haloalkyl, C₁-C₄ hydroxyalkyl, or C₁-C₄ haloalkoxy or hydroxy. Each R^{B}′ independently represents H or C₁-C₆ alkyl.

‘Acyloxy’ refers to the group —OC(O)R²³ where R²³ is hydrogen, alkyl, aryl or cycloalkyl.

‘Alkoxy’ refers to the group —OR²⁴ where R²⁴ is alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like. Particular alkoxy groups are lower alkoxy, i.e. with between 1 and 6 carbon atoms.

‘Substituted alkoxy’ includes those groups recited in the definition of “substituted” herein, and particularly refers to an alkoxy group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, heteroaryl, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

‘Alkyl’ means straight or branched aliphatic hydrocarbon having 1 to about 20 carbon atoms. Preferred alkyl has 1 to about 12 carbon atoms. More preferred is lower alkyl which has 1 to 6 carbon atoms. Most preferred are groups such as methyl, ethyl and propyl. Branched means that one or more lower alkyl groups such as methyl, ethyl or propyl is attached to a linear alkyl chain. The term C₁-C₆ alkyl includes both branched and straight chain groups, exemplary straight chain groups include ethyl, propyl, butyl, exemplary branched chain groups include isopropyl, isoamyl, and the like.

‘Substituted alkyl’ includes those groups recited in the definition of “substituted” herein, and particularly refers to an alkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, heteroaryl, keto, nitro, thioalkoxy, substituted thioalkoxy, thioaryloxy, thioketo, thiol, alkyl-S(O)—, aryl-S(O)—, alkyl-S(O)₂—, and aryl-S(O)₂—.

As used herein, the term “metal” includes and contemplates reactive metals, such as are useful, for example, in catalysis, and metals that are divalent. Exemplary and non-limiting examples of metals contemplated by the present invention, comprise Ag, Au, Co, Cu, Fe, Mn, Ni, Pd, Pt, Rh, Ru, Zn and the like.

When describing the peptoids and peptoid compositions containing such peptoids, the following terms have the following meanings unless otherwise indicated.

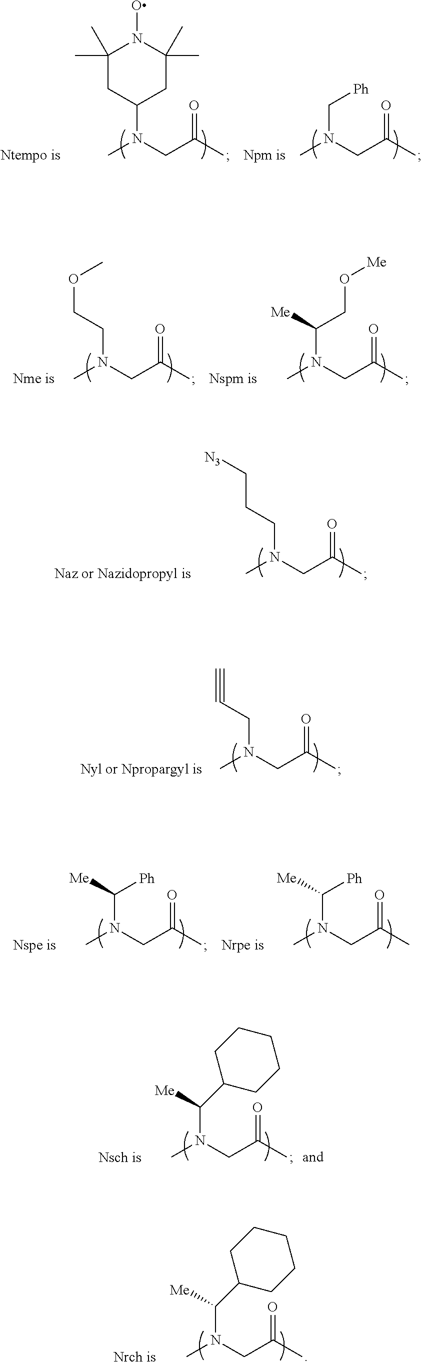

“Unnatural amino acids” means amino acids and corresponding cyclic peptoid units that are synthesized from single amino acid starting materials. Such unnatural amino acids may be prepared and used individually in accordance with the present invention, or may incorporated into existing proteins. This method may be used to create analogs with unnatural amino acids. A general method for site-specific incorporation of unnatural amino acids into proteins is described in Christopher J. Noren, Spencer J. Anthony-Cahill, Michael C. Griffith, Peter G. Schultz, Science, 244:182-188 (April 1989).

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base.

Tautomeric forms may be relevant to the attainment of the optimal chemical reactivity and biological activity of a compound of interest.

As used herein, the term “isotopic variant” refers to a compound that comprises an unnatural proportion of an isotope of one or more of the atoms that constitute such compound. For example, an “isotopic variant” of a compound can comprise an unnatural proportion of one or more non-radioactive isotopes, such as for example, deuterium (²H or D), carbon-13 (¹³C), nitrogen-15 (¹⁵N), or the like. It will be understood that, in a compound comprising an unnatural proportion of an isotope, any example of an atom where present, may vary in isotope composition. For example, any hydrogen may be ²H/D, or any carbon may be ¹³C, or any nitrogen may be ¹⁵N, and that the presence and placement of such atoms may be determined within the skill of the art. Likewise, provided herein are methods for preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ⁵O and ¹³N, and would be useful in Positron Emission Topography (PET) studies for examining substrate receptor occupancy. All isotopic variants of the compounds provided herein, radioactive or not, are intended to be encompassed within the scope provided herein.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

### The Peptoids

As set forth earlier herein, the N-substituted glycine peptoids contain side chains or pendant end groups with chemical functionalities that contribute to catalytic activity. The peptoids may be useful in substrate selective catalytic transformation and asymmetric catalytic transformation. More particularly, the peptoids may be useful in asymmetric catalytic resolution. These peptoids can accordingly include natural/normatural amino acids: beta-amino acids, D-amino acids and/or other proteinogenic and abiotic amino acids.

More particularly, the present invention relates to peptoids, according to formula Ia or Ib:

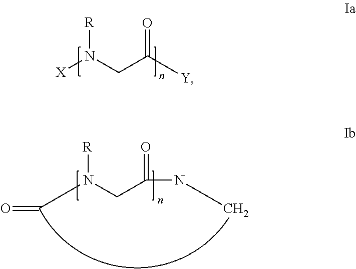

comprised of monomers according to formula II and formula III:

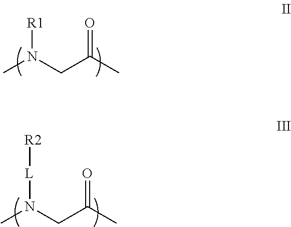

wherein

each R is independently substituted or unsubstituted alkyl, substituted or unsubstituted alkenyl, substituted or unsubstituted alkynyl, substituted or unsubstituted heterocycloalkyl, substituted or unsubstituted aryl, or substituted or unsubstituted heteroaryl;

each R¹ is independently substituted or unsubstituted alkyl, substituted or unsubstituted alkenyl, substituted or unsubstituted alkynyl, substituted or unsubstituted aryl or substituted or unsubstituted heteroaryl;

each R² is a group or substituent capable of contributing to the catalysis of an organic transformation;

L is a single bond, C₁-C₄ alkylene, —C₂-C₄ alkylene-O—, or —C₂-C₄ alkylene-O—C₁-C₄ alkylene-;

X is H, substituted or unsubstituted acyl; Y is NH₂, OH, acylamino, or acyloxy;

and n is an integer between 2-200;

or a salt thereof; and stereoisomers, isotopic variants and tautomers thereof;

provided that:
v) at least one of the monomers is of formula III.;
vi) each R¹ in the peptoid oligomer may be the same or different;
vii) each -L-R² in the peptoid oligomer may be the same or different; and
viii) the peptoid oligomer is other than:

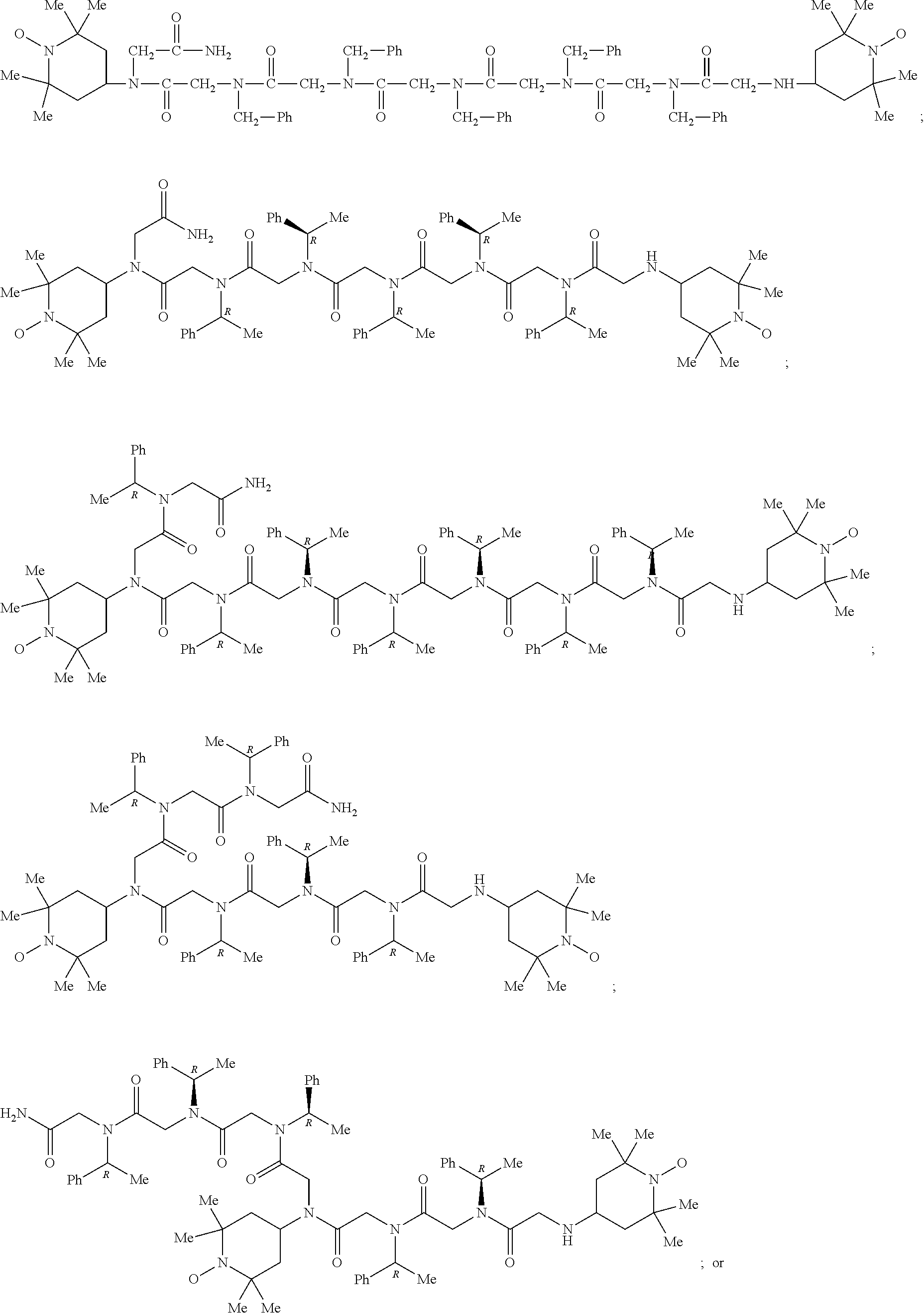

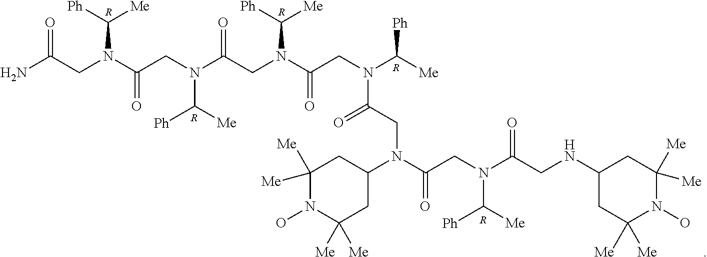

In one embodiment, the invention relates to a peptoid oligomer according to formula Ia or Ib, wherein <20% of the monomers are of formula III at the same time.

In one embodiment the invention relates to a peptoid oligomer according to formula Ia or Ib, wherein 10-60% of the monomers are of formula III at the same time.

In one embodiment, the invention relates to a peptoid oligomer according to formula Ia or Ib, wherein 10-40% of the monomers are of formula III at the same time.

In one embodiment, the invention relates to a peptoid oligomer according to formula Ia or Ib, wherein 10-20% of the monomers are of formula III at the same time.

In one embodiment, with respect to peptoids of formulae Ia-Ib, R¹ is alkyl substituted with phenyl, alkoxy, halo, amino or azido.

In one embodiment with respect to peptoids of formulae Ia-Ib, R¹ is substituted or unsubstituted phenylalkyl.

In one embodiment, with respect to peptoids of formulae Ia-Ib, R¹ is substituted or unsubstituted benzyl.

In one embodiment, with respect to peptoids of formulae Ia-Ib, R¹ is substituted or unsubstituted phenyl.

In one embodiment, with respect to peptoids of formulae Ia-Ib, R¹ is substituted or unsubstituted phenethyl.

In one embodiment, with respect to peptoids of formulae Ia-Ib, R¹ is substituted or unsubstituted phenylpropyl.

In one embodiment, with respect to peptoids of formulae Ia-Ib, R¹ is substituted or unsubstituted naphthylmethyl.

In one embodiment, with respect to peptoids of formulae Ia-Ib, R¹ is substituted or unsubstituted (2-phenyl)phenethyl.

In one embodiment, with respect to peptoids of formulae Ia-Ib, R¹ is substituted or unsubstituted alkoxyalkyl.

In one embodiment, with respect to peptoids of formulae Ia-Ib, R¹ is substituted or unsubstituted methoxyethyl, methoxypropyl, or methoxybutyl.

In one embodiment, with respect to peptoids of formulae Ia-Ib, R¹ is substituted or unsubstituted cycloalkylalkyl.

In one embodiment, with respect to peptoids of formulae Ia-Ib, R¹ is substituted or unsubstituted cycloalkylmethyl.

In one embodiment, with respect to peptoids of formulae Ia-Ib, R¹ is substituted or unsubstituted cyclohexylmethyl, cyclopentylmethyl, cyclobutylmethyl, or cyclopropylmethyl.

In one embodiment, with respect to peptoids of formulae Ia-Ib, R¹ is substituted or unsubstituted alkenyl.

In one embodiment, with respect to peptoids of formulae Ia-Ib, R¹ is substituted or unsubstituted ethenyl, propenyl or butenyl.

In one embodiment, with respect to peptoids of formulae Ia-Ib, R¹ is substituted or unsubstituted alkylnyl.

In one embodiment, with respect to peptoids of formulae Ia-Ib, R¹ is substituted or unsubstituted ethylnyl, propynyl or butynyl.

In one embodiment, with respect to peptoids of formulae Ia-Ib, R¹ is

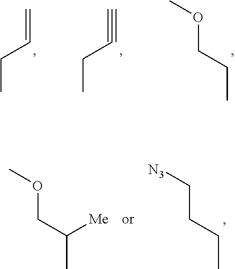

In one embodiment, with respect to peptoids of formulae Ia-Ib, R¹ is

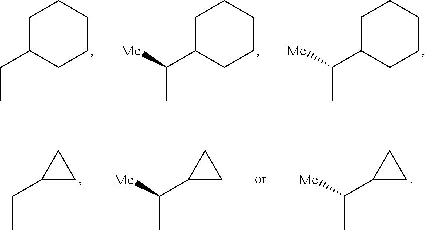

In one embodiment, with respect to peptoids of formulae Ia-Ib, R¹ is

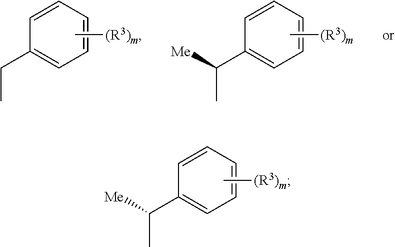

and wherein each R³ is independently alkyl, hydroxy, amino, nitro, or alkoxy and m is 0, 1 or 2.

In one embodiment, with respect to peptoids of formulae Ia-Ib, R¹ is

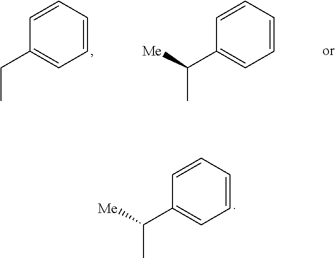

In one embodiment, with respect to peptoids of formulae Ia-Ib, L is a single bond.

In one embodiment, with respect to peptoids of formulae Ia-Ib, L is —CH₂—.

In one embodiment, with respect to peptoids of formulae Ia-Ib, L is —CH₂—O— or CH₂—CH₂—O—.

In one embodiment, with respect to peptoids of formulae Ia-Ib, R² is 8-hydroxyquinolinyl, phenanthrolinyl, terpyridinyl, amino, hydroxyl, carboxy, sulfhydryl, imidazolyl, pyridyl, pyrimidinyl, quinolinyl, or phosphinyl, or metal complexes thereof.

In one embodiment, with respect to peptoids of formulae Ia-Ib, R² is amino, hydroxyl, carboxy, or sulfhydryl or metal complexes thereof.

In one embodiment, with respect to peptoids of formulae Ia-Ib, R² is 8-hydroxyquinolinyl, phenanthrolinyl, terpyridinyl, imidazolyl, pyridyl, or phosphinyl, or metal complexes thereof.

In one embodiment, with respect to peptoids of formulae Ia-Ib, R² is aromatic ketones, or porphyrinyl and metal complexes thereof.

In one embodiment, with respect to peptoids of formulae Ia-Ib, R² is imidazolyl, substituted with one or more groups independently selected from alkyl or halo.

In one embodiment, with respect to peptoids of formulae Ia-Ib, R² is

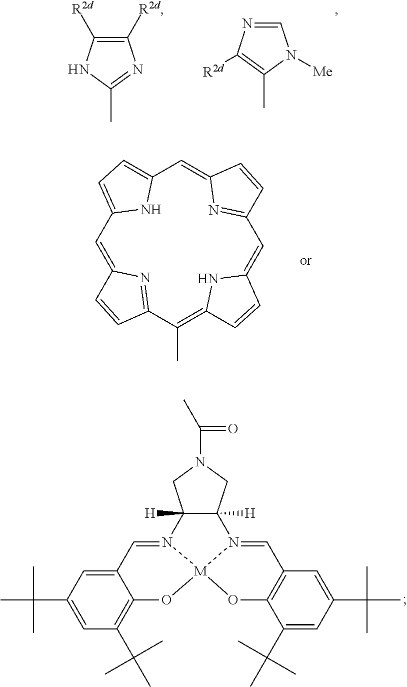

M is Ag, Au, Co, Cu, Fe, Mn, Ni, Pd, Pt, Rh, Ru, or Zn; and R^{2d} is halo, alkyl, or aryl.

In one embodiment, with respect to peptoids of formulae Ia-Ib, R² is —SH, or —CH(Me)NH₂.

In one embodiment, with respect to peptoids of formulae Ia-Ib, R² is a nitroxide containing group.

In one embodiment, with respect to peptoids of formulae Ia-Ib, R² is —C(Me)₂-N(O′)-t-Bu. In another embodiment, R² is —C(Me)₂-N(O′)-Ph.

In one embodiment, with respect to peptoids of formulae Ia-Ib, R² is

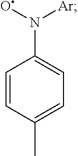

wherein Ar is aryl.

In one embodiment, with respect to peptoids of formulae Ia-Ib, R² is nitroxide containing heterocycloalkyl, or nitroxide containing heteroaryl.

In one embodiment, with respect to peptoids of formulae Ia-Ib, R² is

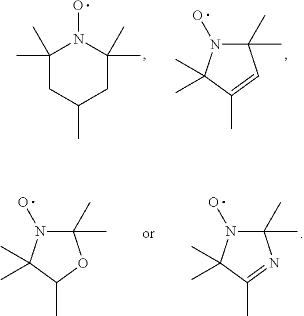

In one embodiment, with respect to peptoids of formulae Ia-Ib, -L-R² is

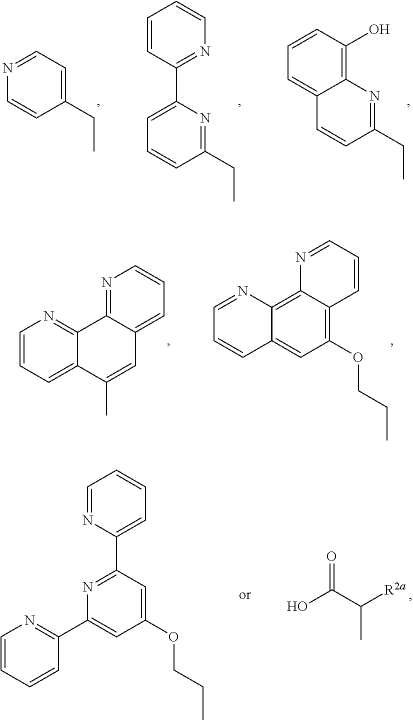

and wherein R^{2a} is substituted or unsubstituted alkyl or aryl.

In one embodiment, with respect to peptoids of formulae Ia-Ib, -L-R² is

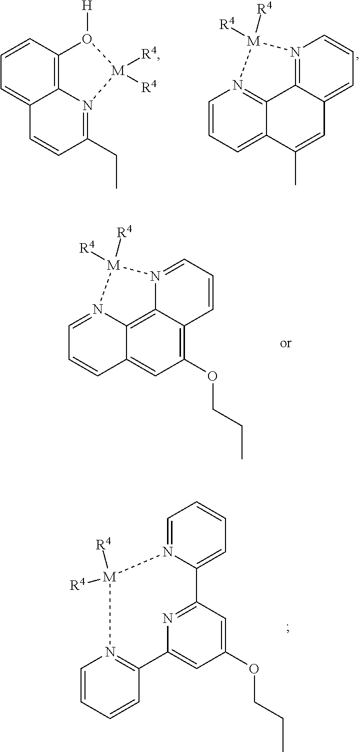

wherein M is Ag, Au, Co, Cu, Fe, Mn, Ni, Pd, Pt, Rh, Ru, or Zn; and R⁴ is Cl, Br, I, alkyl, aryl, hydroxy, SH, SO₃H, SO₂-aryl, or SO₂-alkyl.

In one embodiment, with respect to peptoids of formulae Ia-Ib, -L-R² is

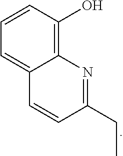

In one embodiment, with respect to peptoids of formulae Ia-Ib, -L-R² is

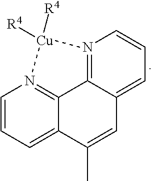

In one embodiment, with respect to peptoids of formulae Ia-Ib, -L-R² is as described in preceding paragraph, and R⁴ is Cl, Br, I, OH, or SH.

In one embodiment, with respect to peptoids of formulae Ia-Ib, -L-R² is as described in preceding paragraph, and R⁴ is SH, SO₃H, SO₂-aryl, or SO₂-alkyl.

In one embodiment, with respect to peptoids of formulae Ia-Ib, -L-R² is as described in preceding paragraph, and R⁴ is Cl.

In one embodiment, with respect to peptoids of formulae Ia-Ib, -L-R² is

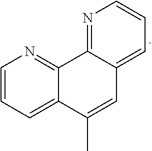

In one embodiment, with respect to peptoids of formulae Ia-Ib, -L-R² is

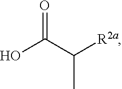

and wherein R^{2a} is substituted or unsubstituted alkyl or aryl.

In one embodiment, with respect to peptoids of formulae Ia-Ib, -L-R² is

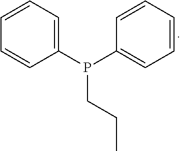 [SMILES: CCCP(c1ccccc1)c1ccccc1]

In one embodiment, with respect to peptoids of formulae Ia-Ib, -L-R² is

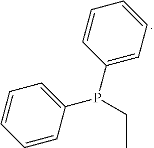

In one embodiment, with respect to peptoids of formulae Ia-Ib, -L-R² is

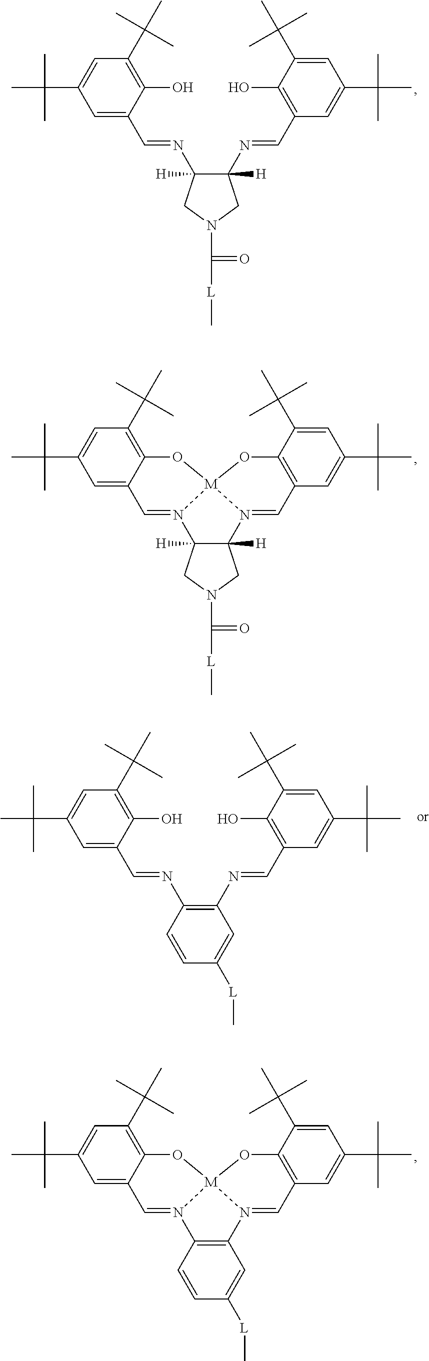

wherein L is a single bond, —CH₂—, —CH(Me)—, —CH₂—CH₂—, or —CH(Me)—CH₂—; and M is a metal. In one embodiment, M is Ag, Au, Co, Cu, Fe, Mn, Ni, Pd, Pt, Rh, Ru, or Zn.

In one embodiment, with respect to peptoids of formulae Ia-Ib, R² is

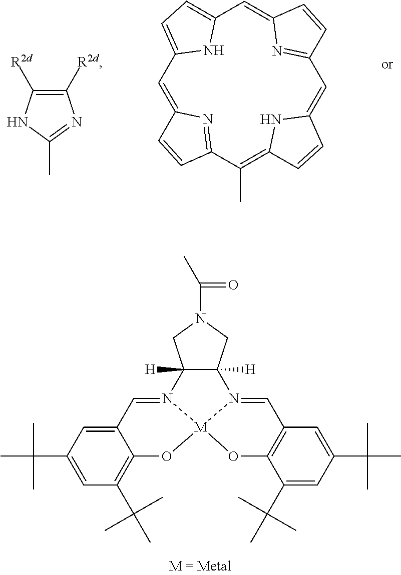

wherein R^{2d} is halo, alkyl or aryl. In one embodiment, M is Ag, Au, Co, Cu, Fe, Mn, Ni, Pd, Pt, Rh, Ru, or Zn.

In one embodiment, with respect to peptoids of formulae Ia-Ib, R² is

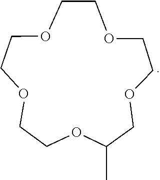 [SMILES: CC1COCCOCCOCCOCCO1]

In one embodiment, with respect to peptoids of formulae Ia-Ib, R² is —SH, or —CH(Me)NH₂.

In one embodiment, with respect to peptoids of formula Ia or Ib, X, Y, R, R¹, R², L and n are as described for formula Ia-Ib; and each monomer of formula II is independently selected from Npm, Nme, Nspm, Naz, Nyl, Nspe, Nrpe, Nsch, and Nrch; and wherein

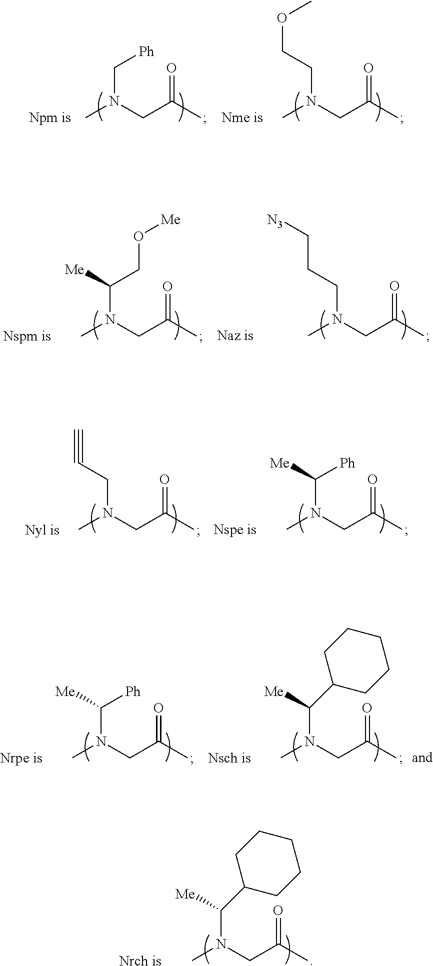

In one embodiment, with respect to peptoids of formulae Ia-Ib, n is 3-100.

In one embodiment, with respect to peptoids of formulae Ia-Ib, n is 3-60.

In one embodiment, with respect to peptoids of formulae Ia-Ib, n is 3-40.

In one embodiment, with respect to peptoids of formulae Ia-Ib, n is 3-20.

In one embodiment, with respect to peptoids of formulae Ia-Ib, n is 4-15.

In one embodiment, with respect to peptoids of formulae Ia-Ib, n is 4-11.

In one embodiment, with respect to acyclic peptoids of formula Ia, X is H or Ac.

In one embodiment, with respect to acyclic peptoids of formula Ia, X is H.

In one embodiment, with respect to acyclic peptoids of formula Ia, Y is OH or OAc.

In one embodiment, with respect to acyclic peptoids of formula Ia, Y is NH₂ or NHAc.

In one embodiment, with respect to acyclic peptoids of formula Ia, Y is NH₂.

In one embodiment, with respect to acyclic peptoids of formula Ia, Y is NHAc.

In one embodiment, with respect to acyclic peptoids of formula Ia, Y is OH.

In one embodiment, with respect to acyclic peptoids of formula Ia, Y is OAc.

In one embodiment, with respect to acyclic peptoids of formula Ia, n is 2-11; one monomer is of formula III; and the other monomers are independently selected from Npm, Nme, Nspm, Naz, Nyl, Nspe, Nrpe, Nsch, and Nrch.

In one embodiment, with respect to acyclic peptoids of formula Ia, n is 7; one monomer is of formula III and the other monomers are independently selected from Npm, Nme, Nspm, Naz, Nyl, Nspe, Nrpe, Nsch, and Nrch.

In one embodiment, with respect to acyclic peptoids of formula Ia, n is 7; and the peptoid is H—N(L-R²)CH₂C(O)—(Nspe)₆-NH₂.

In one embodiment, with respect to acyclic peptoids of formula Ia, n is 7; and the peptoid is H-(Nspe)₃-N(L-R²)CH₂C(O)—(Nspe)₃-NH₂.

In one embodiment, with respect to acyclic peptoids of formula Ia, n is 7; and the peptoid is H-(Nspe)-(Npm)-Nspe-N(L-R²)CH₂C(O)—Nspe-Npm-Nspe-NH₂.

In one embodiment, with respect to acyclic peptoids of formula Ia, n is 7; and the peptoid is H-(Nspe)-(Npm)₂-N(L-R²)CH₂C(O)—(Npm)-2-Nspe-NH₂.

In one embodiment, with respect to acyclic peptoids of formula Ia, n is 7; and the peptoid is H—N(L-R²)CH₂C(O)—(Nspe)₆-NH₂.

In one embodiment, with respect to acyclic peptoids of formula Ia, n is 7; and the peptoid is H—N(L-R²)CH₂C(O)—Nspe-Npm-(Nspe)-2-Npm-Nspe-NH₂.

In one embodiment, with respect to acyclic peptoids of formula Ia, n is 7; and the peptoid is H—N(L-R²)CH₂C(O)—Nrpe-Npm-(Nrpe)-2-Npm-Nrpe-NH₂.

In one embodiment, with respect to acyclic peptoids of formula Ia, n is 7; and the peptoid is H—N(L-R²)CH₂C(O)—(Npm)-2-Nspe-(Npm)-2-Nspe-NH₂.

In one embodiment, with respect to acyclic peptoids of formula Ia, n is 6; and the peptoid is H—N(L-R²)CH₂C(O)— (Nspe)ₛ-NH₂.

In one embodiment, with respect to acyclic peptoids of formula Ia, n is 5; and the peptoid is H—N(L-R²)CH₂C(O)—(Nspe)₄-NH₂.

In one embodiment, with respect to acyclic peptoids of formula Ia, n is 4; and the peptoid is H—N(L-R²)CH₂C(O)— (Nspe)₃-NH₂.

In one embodiment, with respect to acyclic peptoids of formula Ia, n is 3; and the peptoid is H—N(L-R²)CH₂C(O)— (Nspe)₂-NH₂.

In one embodiment, with respect to acyclic peptoids of formula Ia, n is 7; and the peptoid is H-(Npm)₃-N(L-R²)CH₂C(O)— (Npm)₃-NH₂.

In one embodiment, with respect to acyclic peptoids of formula Ia, n is 7; and the peptoid is H—N(L-R²)CH₂C(O)— (Npm)₆-NH₂.

In one embodiment, with respect to acyclic peptoids of formula Ia, n is 7; and the peptoid is H—N(L-R²)CH₂C(O)—NrpeNpm(Nrpe)₂NpmNrpe-NH₂.

In one embodiment, with respect to acyclic peptoids of formula Ia, n is 7; and the peptoid is H—N(L-R²)CH₂C(O)— (Nspe)₃(Nrpe)₃-NH₂.

In one embodiment, with respect to acyclic peptoids of formula Ia, n is 4; and the peptoid is H—N(L-R²)CH₂C(O)— (Nsmp)₃-NH₂.

In one embodiment, with respect to acyclic peptoids of formula Ia, n is 7; and the peptoid is H—N(L-R²)CH₂C(O)—NsmpNme(Nsmp)₂NmeNsmp-NH₂.

In one embodiment, with respect to acyclic peptoids of formula Ia, n is 6; and the peptoid is H-NspeNaz-N(L-R²)CH₂C(O)—NspeNylNspe-NH₂.

In one embodiment, with respect to acyclic peptoids of formula Ia, n is 7; and the peptoid is H-Naz(Nspe)₂-N(L-R²)CH₂C(O)—NspeNylNspe-NH₂.

In one embodiment, with respect to acyclic peptoids of formula Ia, n is 9; and the peptoid is H-(Nspe)₄-N(L-R²)CH₂C(O)— (Nspe)₄-NH₂.

In one embodiment, with respect to acyclic peptoids of formula Ia, n is 7; and the peptoid is H—N(L-R²)CH₂C(O)— (Nspe)₃(Npm)₃-NH₂.

In one embodiment, with respect to the cyclic peptoids of formula Ib, the peptoid is

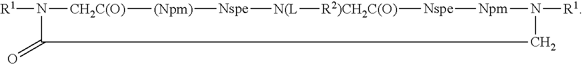

In one embodiment, with respect to the cyclic peptoids of formula Ib, the peptoid is

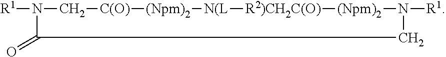

In one embodiment, with respect to the cyclic peptoids of formula Ib, the peptoid is

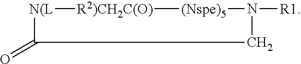

In one embodiment, with respect to the cyclic peptoids of formula Ib, the peptoid is

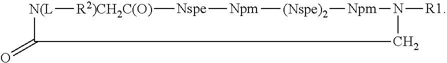

In one embodiment, with respect to the cyclic peptoids of formula Ib, the peptoid is

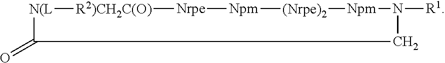

In one embodiment, with respect to the cyclic peptoids of formula Ib, the peptoid is

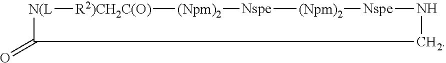

In one embodiment, with respect to the cyclic peptoids of formula Ib, the peptoid is

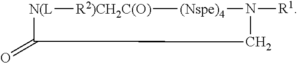

In one embodiment, with respect to the cyclic peptoids of formula Ib, n is 4; and the peptoid is

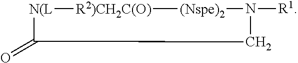

In one embodiment, with respect to the cyclic peptoids of formula Ib, the peptoid is

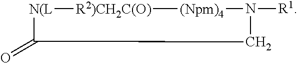

In one embodiment, with respect to the cyclic peptoids of formula Ib, the peptoid is

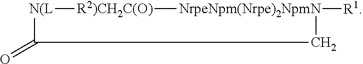

In one embodiment, with respect to the cyclic peptoids of formula Ib, the peptoid is

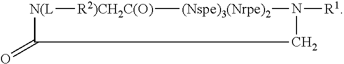

In one embodiment, with respect to the cyclic peptoids of formula Ib, the

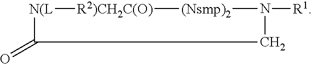

In one embodiment, with respect to the cyclic peptoids of formula Ib, the peptoid is

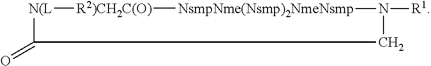

In one embodiment, with respect to the cyclic peptoids of formula Ib, the peptoid is

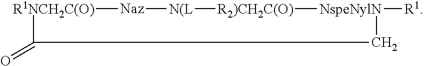

In one embodiment, with respect to the cyclic peptoids of formula Ib, the peptoid is

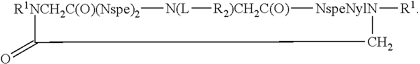

In one embodiment, with respect to the cyclic peptoids of formula Ib, the peptoid is

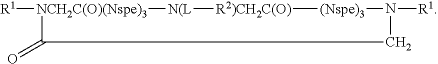

In one embodiment, with respect to the cyclic peptoids of formula Ib, the peptoid is

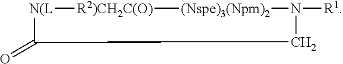

In one embodiment, with respect to the cyclic peptoids of formula Ib, the peptoid is as depicted in the preceding paragraphs; and R¹ is

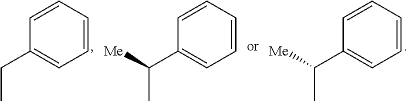

In one embodiment, with respect to the cyclic peptoids of formula Ib, the peptoid is as depicted in the preceding paragraphs; and R¹ is

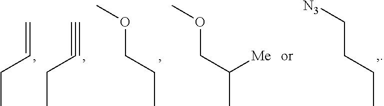

In one embodiment, with respect the peptoids depicted in the preceding paragraphs, L is a single bond; and L-R² is

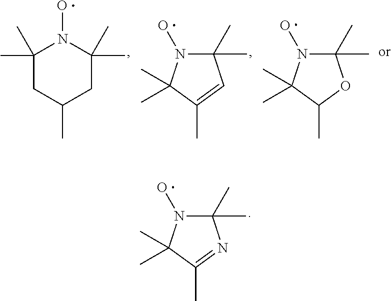

In one embodiment, with respect to the peptoids depicted in the preceding paragraphs, L is a single bond; and L-R² is

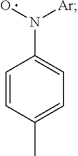

wherein Ar is substituted or unsubstituted aryl. In one embodiment, Ar is substituted or unsubstituted phenyl.

In one embodiment, with respect to the peptoids described in the preceding paragraphs, -L-R² is

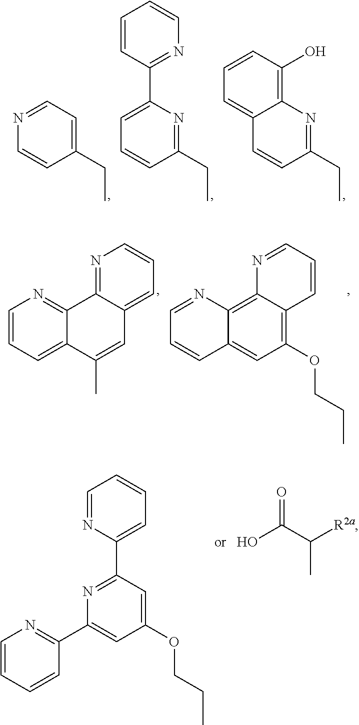

and wherein R^{2a} is substituted or unsubstituted alkyl or aryl.

In one embodiment, with respect to the peptoids described in the preceding paragraphs, -L-R² is

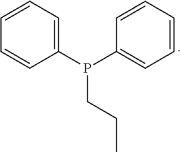 [SMILES: CCCP(c1ccccc1)c1ccccc1]

In one embodiment, with respect to the peptoids described in the preceding paragraphs, -L-R² is

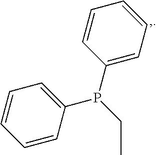

In one embodiment, with respect to the peptoids described in the preceding paragraphs, -L-R² is

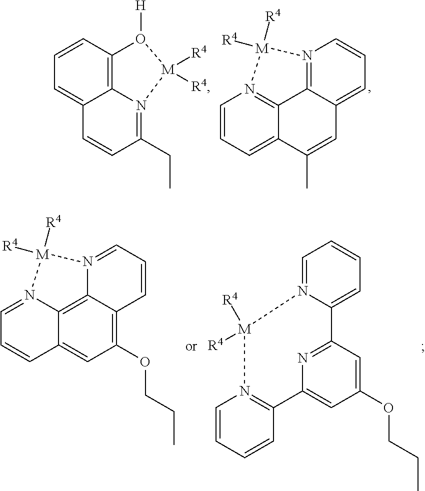

wherein M is Ag, Au, Co, Cu, Fe, Mn, Ni, Pd, Pt, Rh, Ru, or Zn; and R⁴ is Cl, Br, I, alkyl, aryl, hydroxy, SH, SO₃H, SO₂-aryl, or SO₂-alkyl.

In one embodiment, with respect to the peptoids described in the preceding paragraphs, -L-R² is

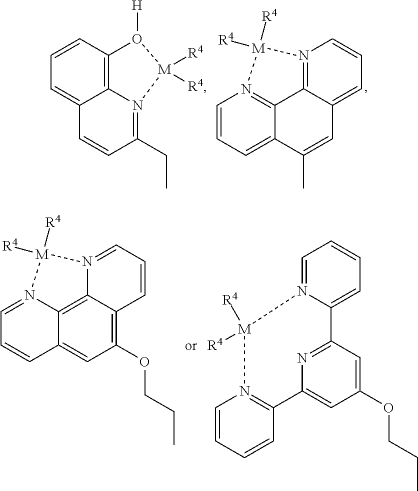

and wherein M is Ag, Au, Co, Cu, Fe, Mn, Ni, Pd, Pt, Rh, Ru, or Zn ; and R⁴ is Cl.

In one embodiment, with respect to peptoids of formula Ia, the peptoid is selected from:

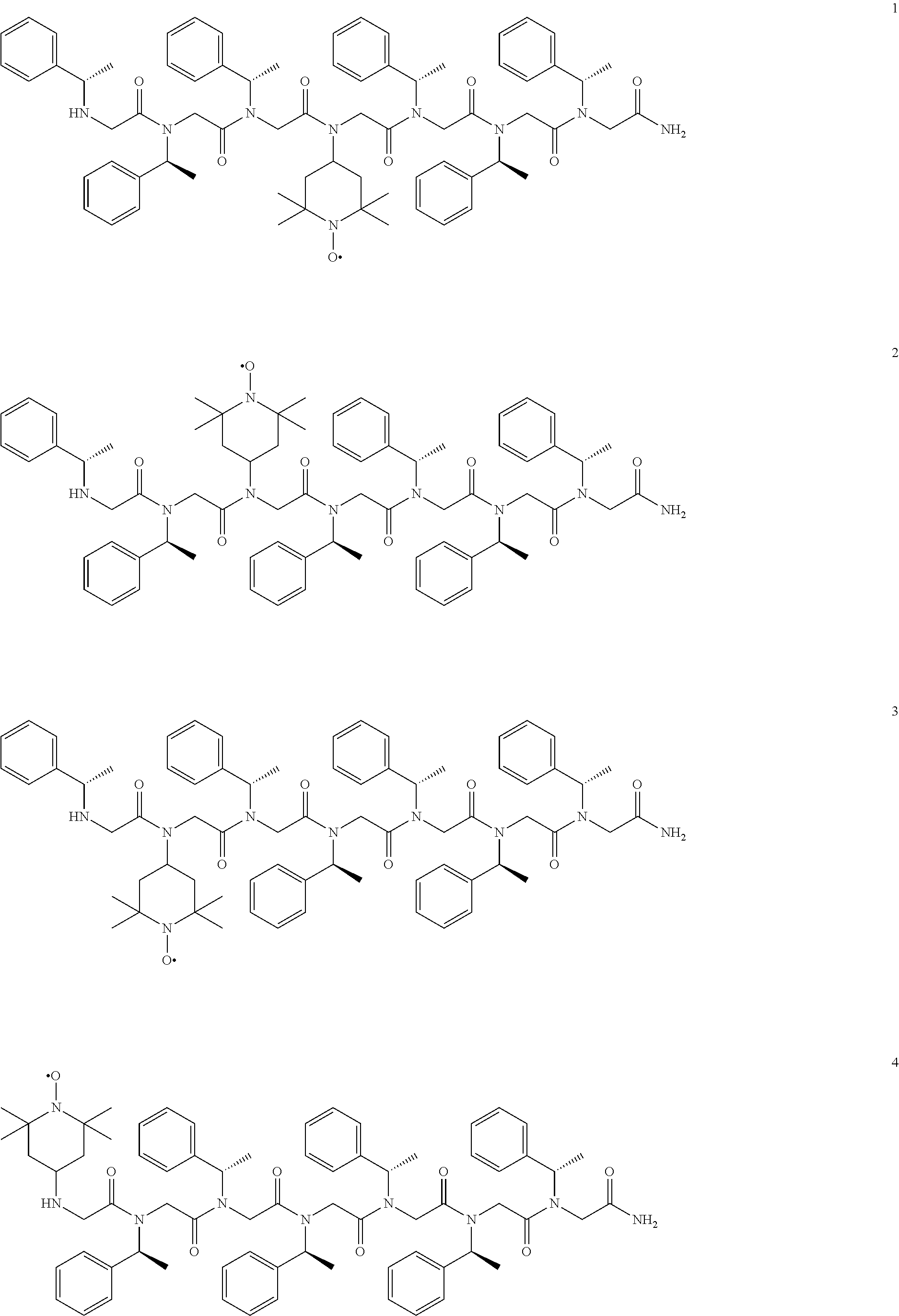

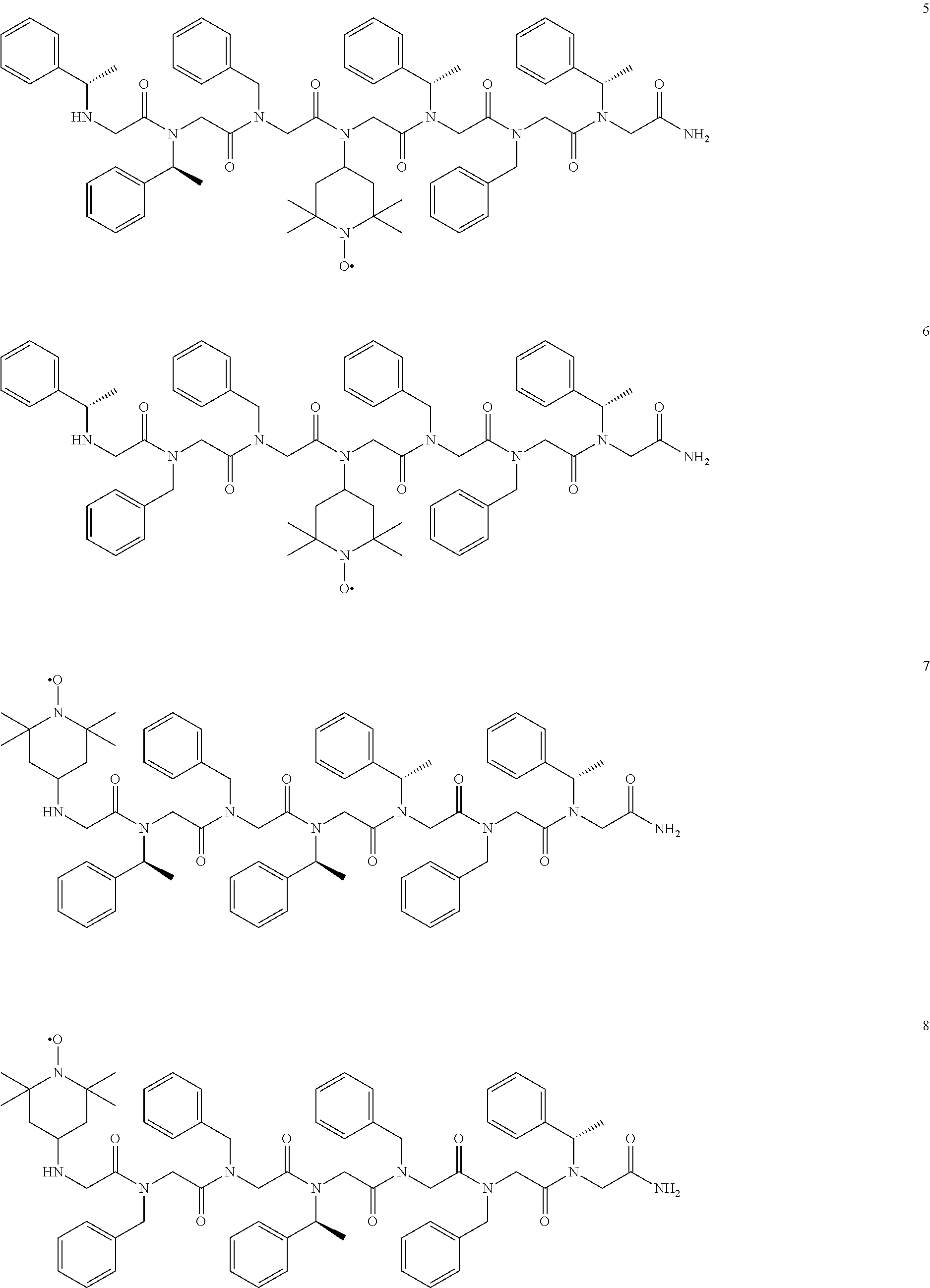

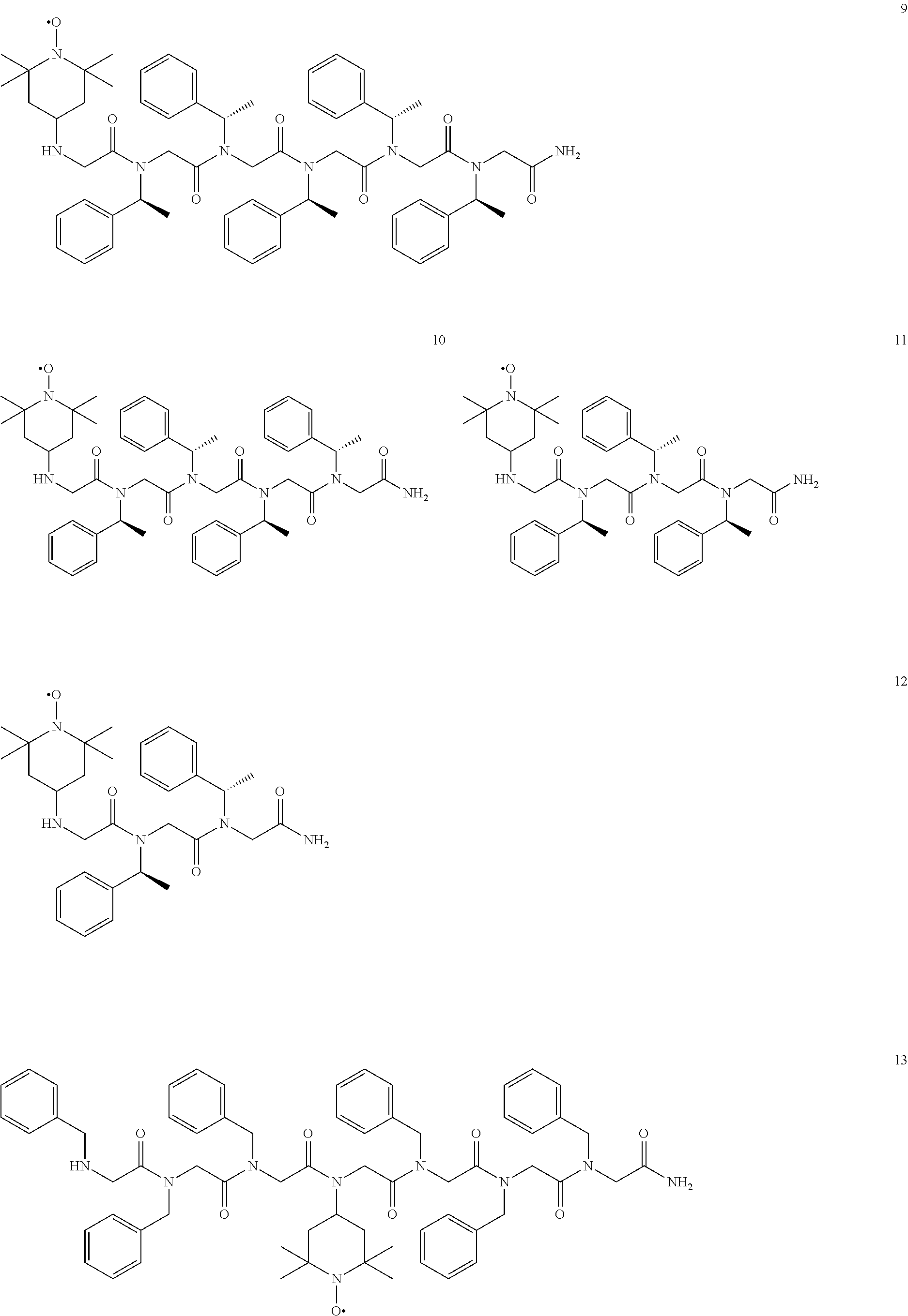

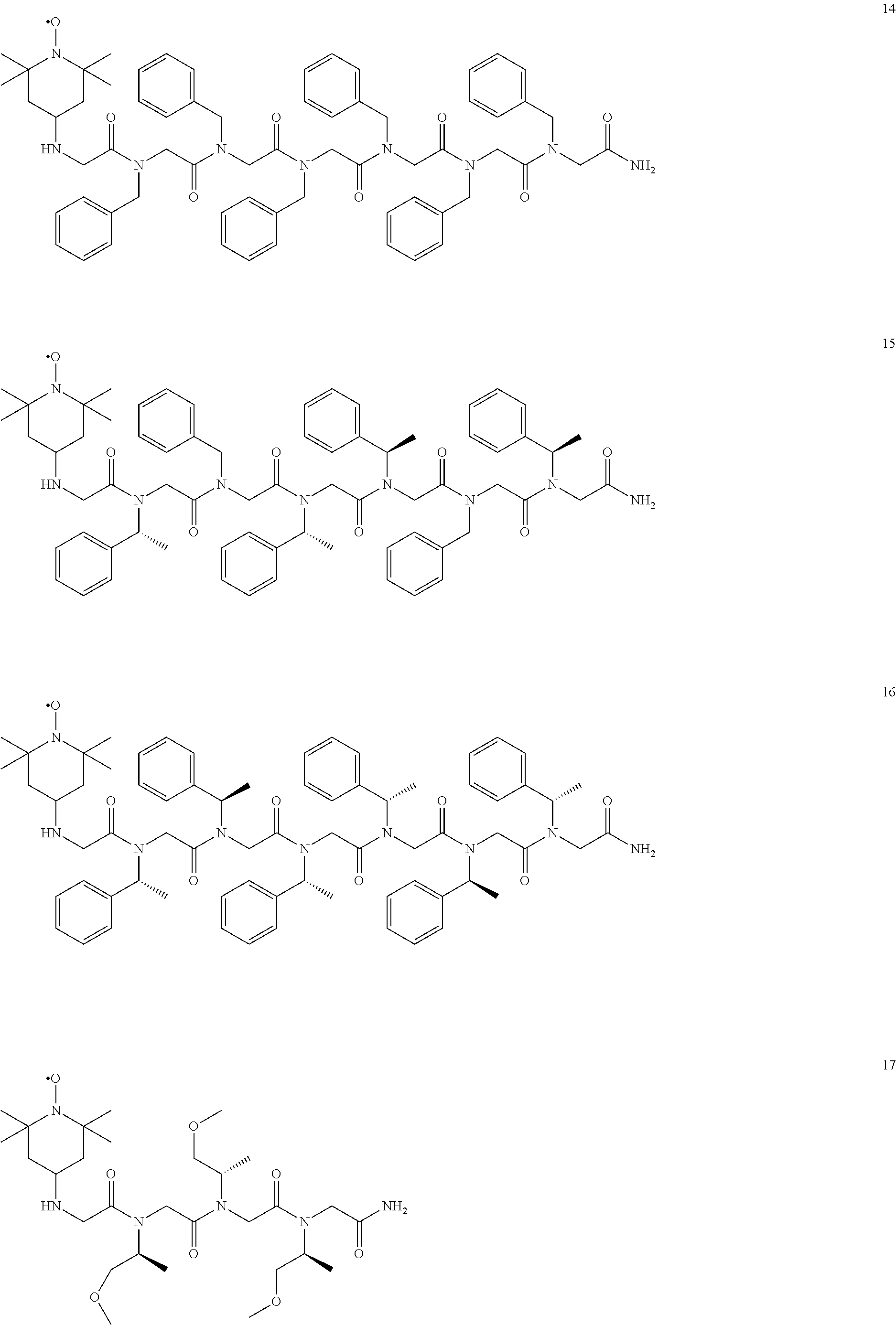

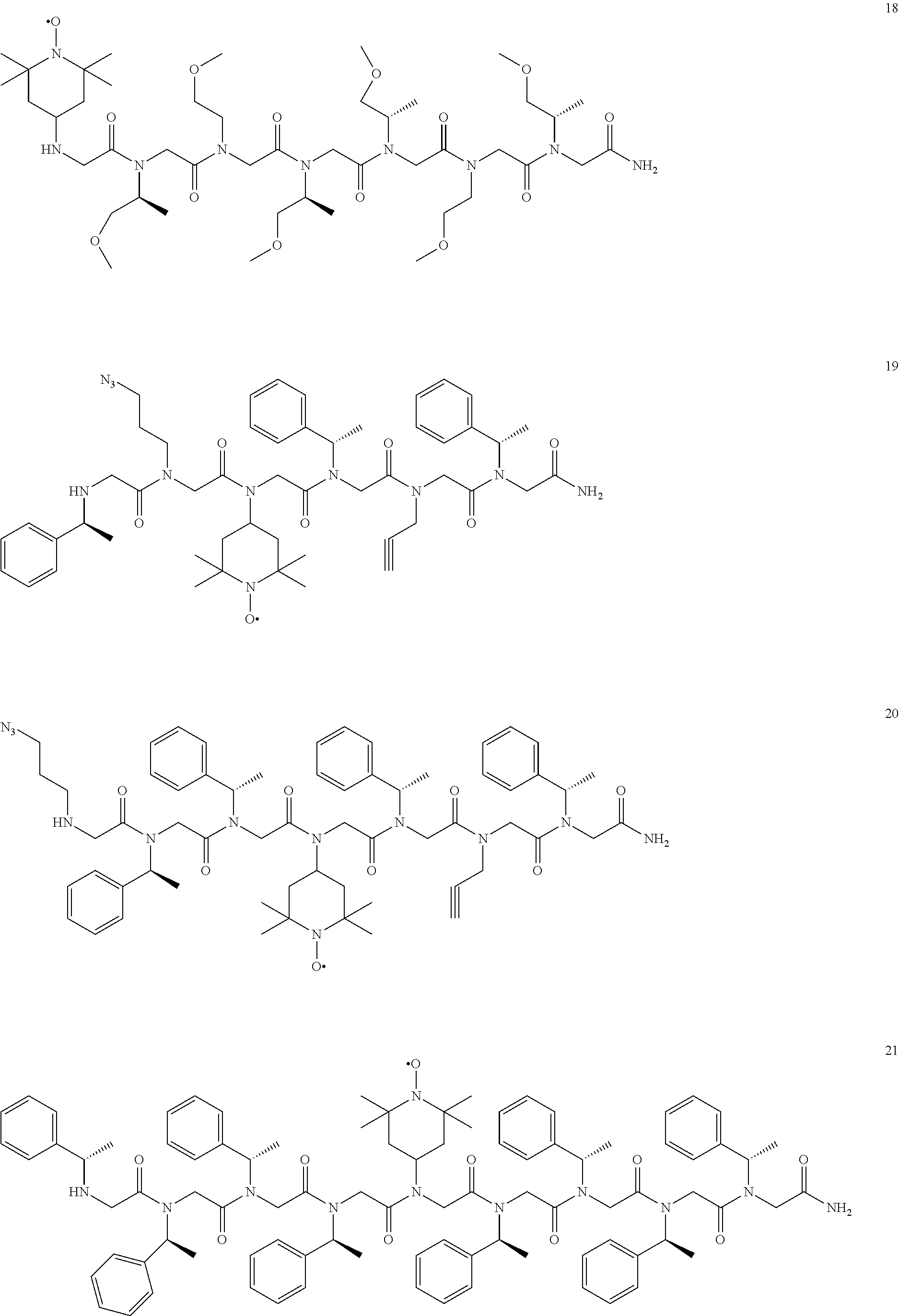

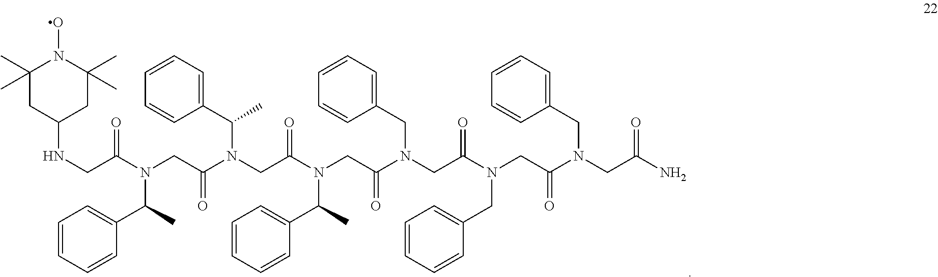

In one embodiment, with respect to acyclic peptoids of formula Ia, the peptoid is selected from:

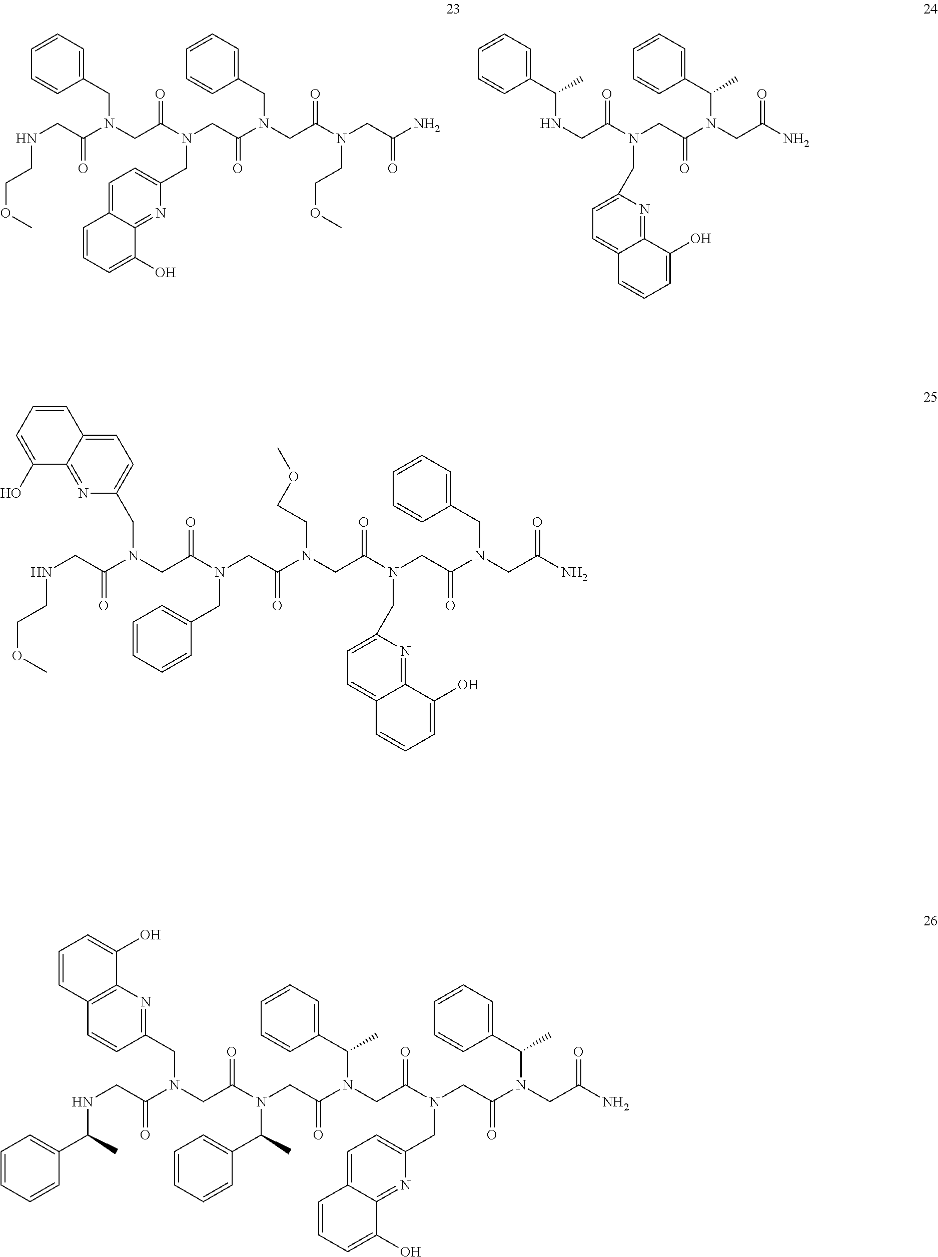 [SMILES: COCCNCC(=O)N(CC(=O)N(CC(=O)N(CC(=O)N(CCOC)CC(N)=O)Cc1ccccc1)Cc1ccc2cccc(O)c2n1)Cc1ccccc1 ; C[C@H](NCC(=O)N(CC(=O)N(CC(N)=O)[C@@H](C)c1ccccc1)Cc1ccc2cccc(O)c2n1)c1ccccc1 ; COCCNCC(=O)N(CC(=O)N(CC(=O)N(CCOC)CC(=O)N(CC(=O)N(CC(N)=O)Cc1ccccc1)Cc1ccc2cccc(O)c2n1)Cc1ccccc1)Cc1ccc2cccc(O)c2n1 ; C[C@H](NCC(=O)N(CC(=O)N(CC(=O)N(CC(=O)N(CC(=O)N(CC(N)=O)[C@@H](C)c1ccccc1)Cc1ccc2cccc(O)c2n1)[C@@H](C)c1ccccc1)[C@@H](C)c1ccccc1)Cc1ccc2cccc(O)c2n1)c1ccccc1]

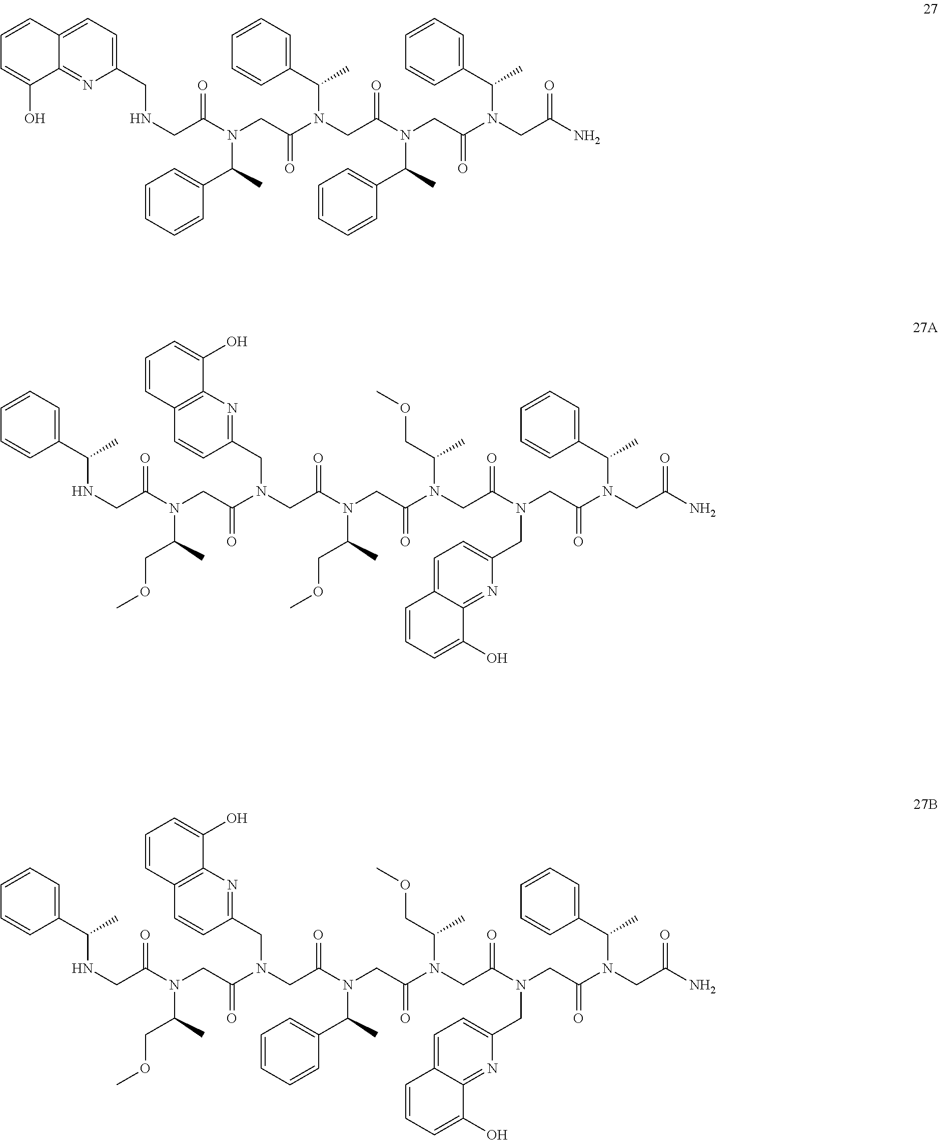 [SMILES: C[C@@H](c1ccccc1)N(CC(N)=O)C(=O)CN(C(=O)CN(C(=O)CN(C(=O)CNCc1ccc2cccc(O)c2n1)[C@@H](C)c1ccccc1)[C@@H](C)c1ccccc1)[C@@H](C)c1ccccc1 ; COC[C@H](C)N(CC(=O)N(CC(=O)N(CC(=O)N(CC(=O)N(CC(=O)N(CC(N)=O)[C@@H](C)c1ccccc1)Cc1ccc2cccc(O)c2n1)[C@@H](C)COC)[C@@H](C)COC)Cc1ccc2cccc(O)c2n1)C(=O)CN[C@@H](C)c1ccccc1 ; COC[C@H](C)N(CC(=O)N(CC(=O)N(CC(=O)N(CC(=O)N(CC(=O)N(CC(N)=O)[C@@H](C)c1ccccc1)Cc1ccc2cccc(O)c2n1)[C@@H](C)COC)[C@@H](C)c1ccccc1)Cc1ccc2cccc(O)c2n1)C(=O)CN[C@@H](C)c1ccccc1]

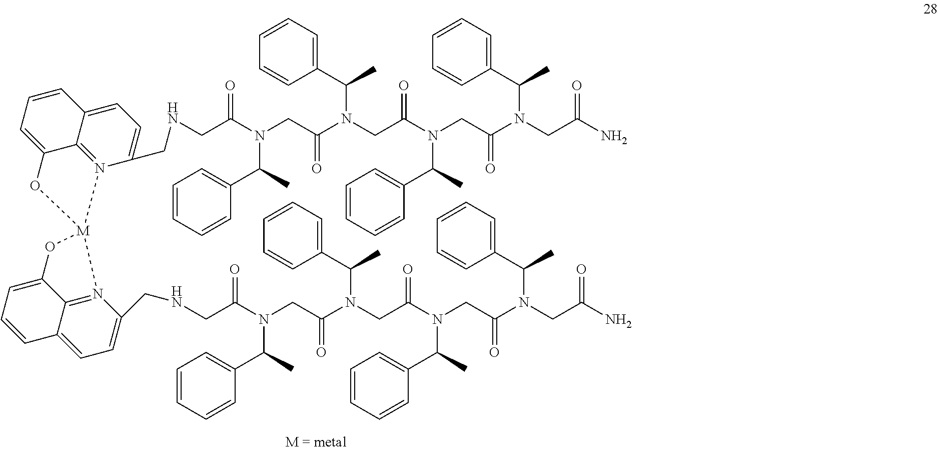

In one embodiment, with respect to acyclic peptoids of formula Ia, the peptoid is selected from:

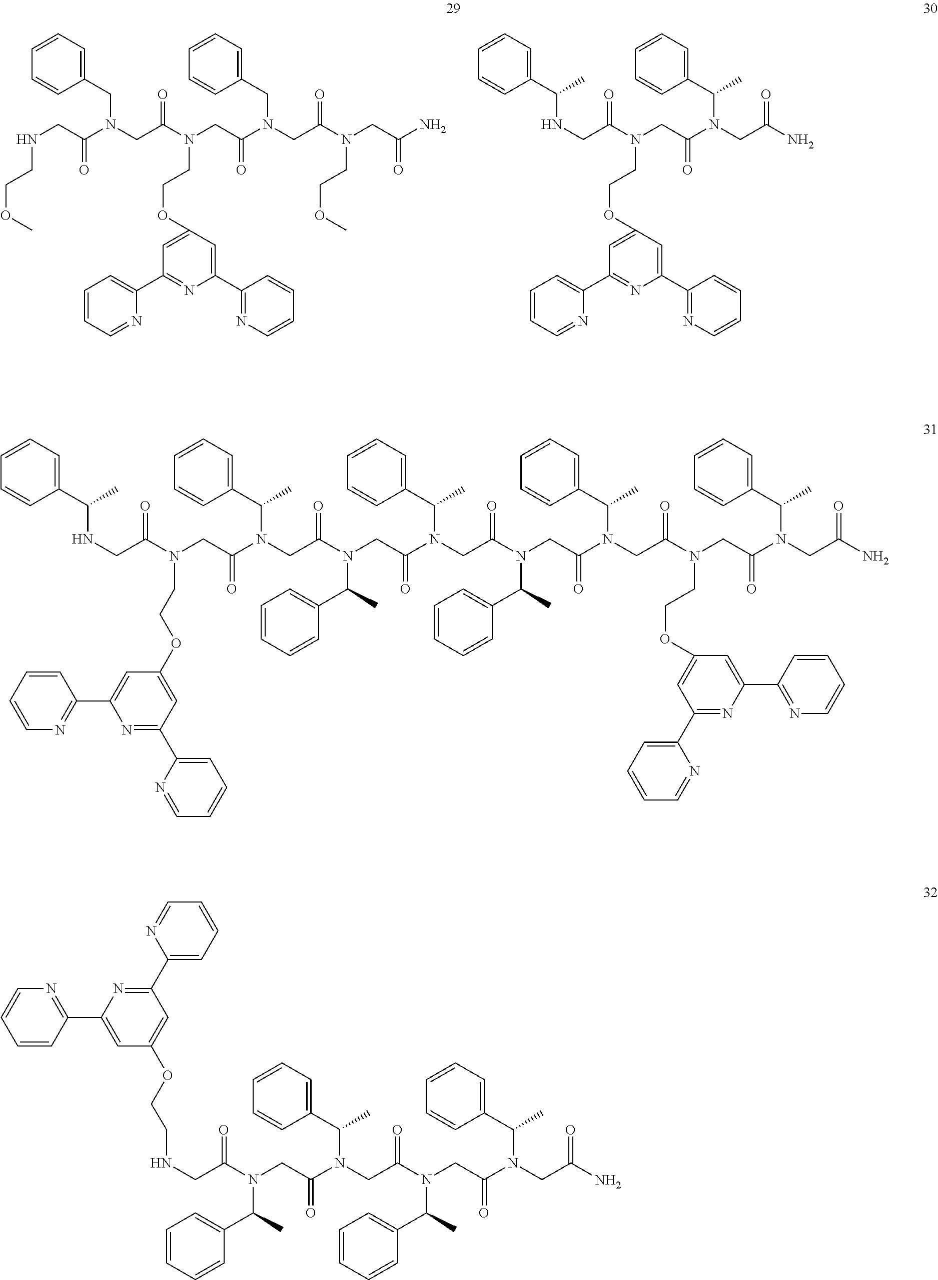

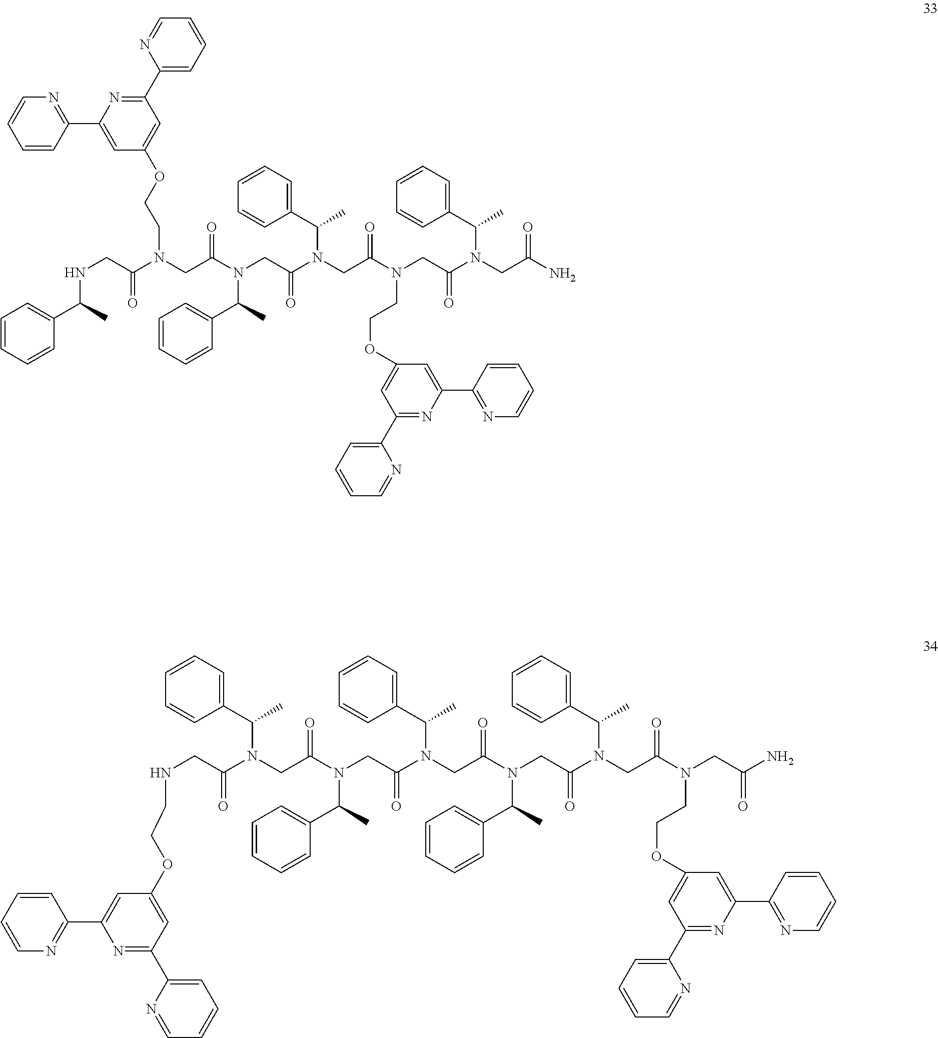 [SMILES: C[C@H](NCC(=O)N(CCOc1cc(-c2ccccn2)nc(-c2ccccn2)c1)CC(=O)N(CC(=O)N(CC(=O)N(CCOc1cc(-c2ccccn2)nc(-c2ccccn2)c1)CC(=O)N(CC(N)=O)[C@@H](C)c1ccccc1)[C@@H](C)c1ccccc1)[C@@H](C)c1ccccc1)c1ccccc1 ; C[C@@H](c1ccccc1)N(CC(=O)N(CCOc1cc(-c2ccccn2)nc(-c2ccccn2)c1)CC(N)=O)C(=O)CN(C(=O)CN(C(=O)CN(C(=O)CN(C(=O)CNCCOc1cc(-c2ccccn2)nc(-c2ccccn2)c1)[C@@H](C)c1ccccc1)[C@@H](C)c1ccccc1)[C@@H](C)c1ccccc1)[C@@H](C)c1ccccc1]

In one embodiment, with respect to acyclic peptoids of formula Ia, the peptoid is selected from:

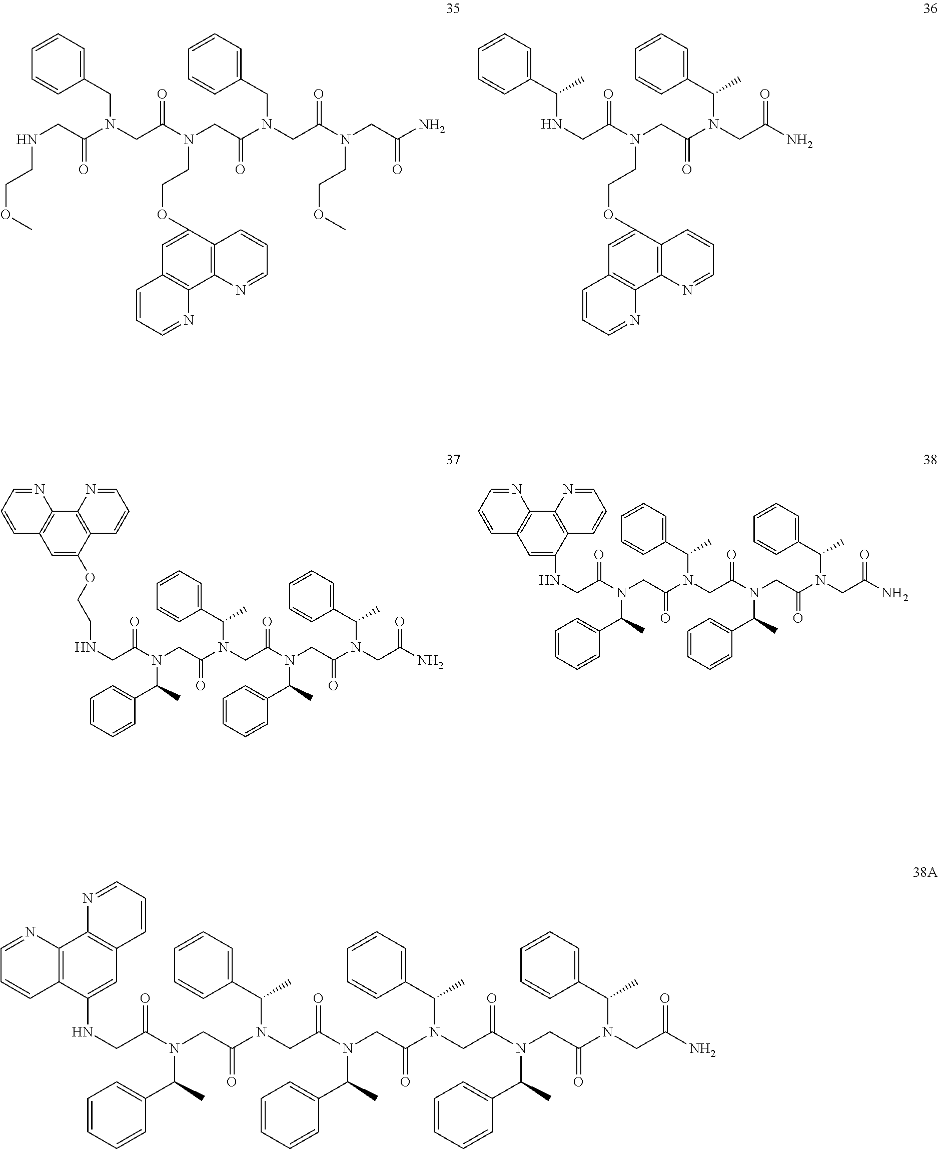

In one embodiment, with respect to peptoids of formula Ia-Ib, the peptoid is selected from:

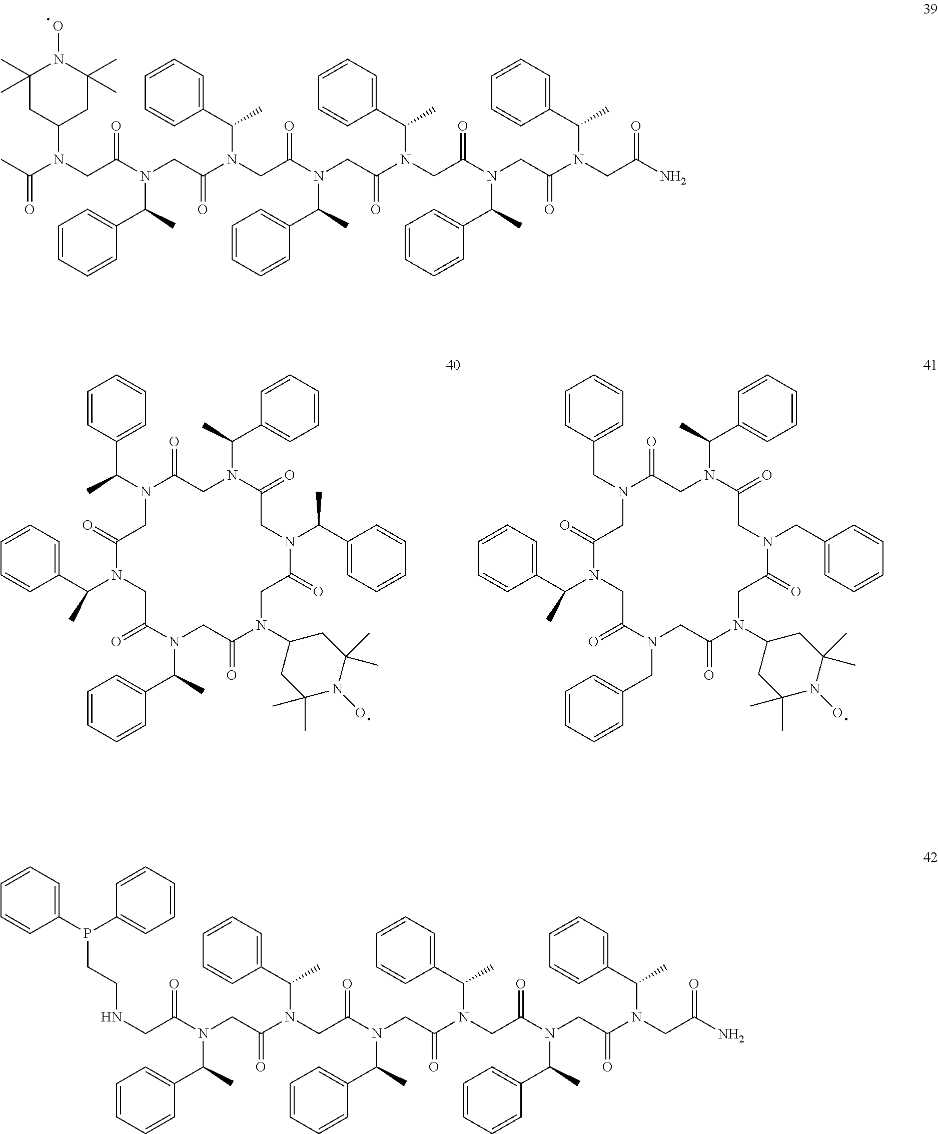

In one embodiment, with respect to acyclic peptoids of formula Ia, the peptoid is selected from:
X-(Nspe)₃Ntempo(Nspe)₃-Y
X-(Nspe)₂Ntempo(Nspe)₄-Y
X-NspeNtempo(Nspe)₅-Y
X-Ntempo(Nspe)₆-Y
X-Ntempo(Nrpe)₆-Y
X-(Nspe)₂ NpmNtempoNspeNpmNspe-Y
X-Nspe(Npm)₂Ntempo(Npm)₂ Nspe-Y
X-NtempoNspeNpm(Nspe)₂ NpmNspe-Y
X-Ntempo(Npm)₂ Nspe(Npm)₂ Nspe-Y
X-Ntempo(Nspe)₅-Y
X-Ntempo(Nspe)₄-Y
X-Ntempo(Nspe)₃-Y
X-Ntempo(Nspe)₂-Y
X-(Npm)₃Ntempo(Npm)₃-Y
X-Ntempo(Npm)₆-Y
X-NtempoNrpeNpm(Nrpe)₂NpmNrpe-Y
X-Ntempo(Nspe)₃(Nrpe)₃-Y
X-Ntempo(Nsmp)₃-Y
X-NtempoNsmpNme(Nsmp)₂NmeNsmp-Y
X-NspePropylazideNtempoNspePropagylNspe-Y
X-Propylazi de(Nspe)₂NtempoNspePropagylNspe-Y
X-(Nspe)₄Ntempo(Nspe)₄-Y
X-Ntempo(Nspe)₃(Npm)₃-Y and
X-NspeNpmNspeNtempoNspeNpmNspe-Y
   and wherein X, and Y are as described for formula I; and Npm, Nme, Nspm, Naz, Nyl, Nspe, Nrpe, Nsch, and Nrch are as defined herein.

In one embodiment, with respect to acyclic peptoids described in preceding paragraph, X is H or Ac.

In one embodiment, with respect to acyclic peptoids described in preceding paragraph, X is H.

In one embodiment, with respect to acyclic peptoids described in preceding paragraph, Y is OH or OAc.

In one embodiment, with respect to acyclic peptoids described in preceding paragraph, Y is NH₂ or NHAc.

In one embodiment, with respect to acyclic peptoids described in preceding paragraph, Y is NH₂.

In one embodiment, with respect to acyclic peptoids described in preceding paragraph, Y is NHAc.

In one embodiment, with respect to acyclic peptoids described in preceding paragraph, Y is OH.

In one embodiment, with respect to acyclic peptoids described in preceding paragraph, Y is OAc.

In a further aspect, the peptoids of the invention may be prepared with a variety of catalytic moieties, including reactive metals such as Ag, Au, Co, Cu, Fe, Mn, Ni, Pd, Pt, Rh, Ru, Zn and the like.

In a yet further aspect, the present invention provides use of the peptoid of the invention as a catalyst in an asymmetric catalytic transformation.

In a further aspect, the present invention provides use of the peptoid of the invention as a catalyst in an asymmetric catalytic resolution.

In a further aspect, the present invention provides use of the peptoid of the invention as a catalyst in regio-selective catalytic transformation.

In a further aspect, the present invention provides use of the peptoid of the invention as a catalyst in a synthesis of enantiomerically pure organic compounds.

In a further aspect, the present invention provides use of the peptoid of the invention as a catalyst in a asymmetric catalytic resolution of aromatic secondary alcohols.

In a further aspect, the present invention provides use of the peptoid of the invention as a catalyst in hydrolysis, aldol reaction, aldol condensation, Diels-Alder reaction, electrochemical oxidation, Michael reaction, epoxidation, hydrogenation, acylation and phosphorylation.

In a further aspect, the present invention provides use of the peptoid of the invention as a catalyst in regioselective and enantioselective nucleophilic transfer reactions

In a further aspect, the present invention provides use of the peptoid of the invention as a catalyst in Baeyer-Villiger oxidation of carbonyl groups to esters.

In a further aspect, the present invention provides use of the peptoid of the invention as a catalyst in solution phase or heterogeneous catalytic transformation.

In a further aspect, the present invention provides use of the peptoid of the invention as a catalyst in regio-selective acylation of polyols.

In a further aspect, the present invention provides use of the peptoid of the invention as a catalyst in regio-selective acylation of tetraols.

In a further aspect, the present invention provides use of the peptoid of the invention as a catalyst in regio-selective acylation of triols.

In a further aspect, the present invention provides use of the peptoid of the invention as a catalyst in regio-selective acylation of diols.

### General Synthetic Procedures

The complexes of this invention can be prepared from readily available starting materials using the general methods and procedures described earlier and illustrated schematically in the examples that follow. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The following methods are presented with details as to the preparation of representative cyclic peptoids that have been listed hereinabove. The cyclic peptoids of the invention may be prepared from known or commercially available starting materials and reagents by one skilled in the art of organic synthesis.

### General Peptoid Synthesis Protocol

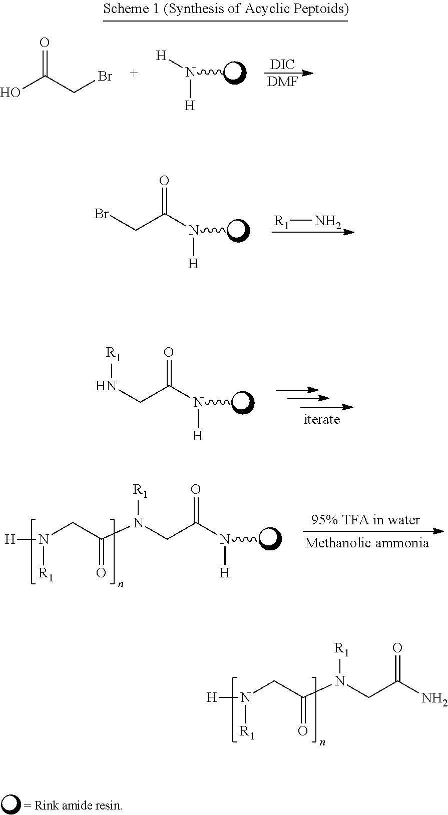

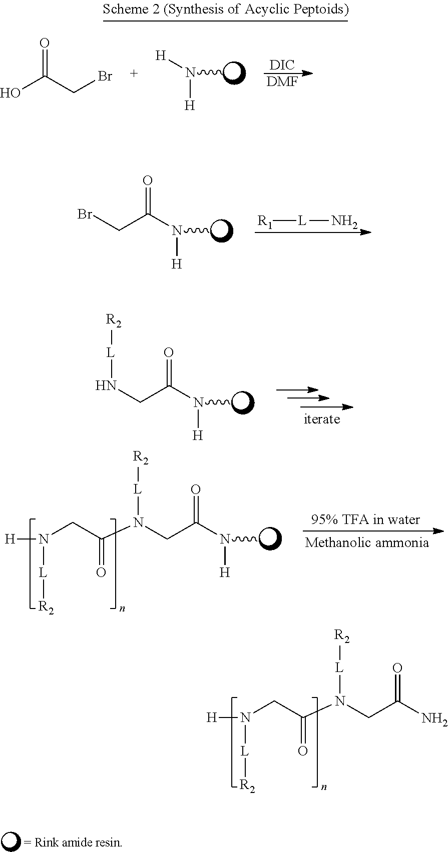

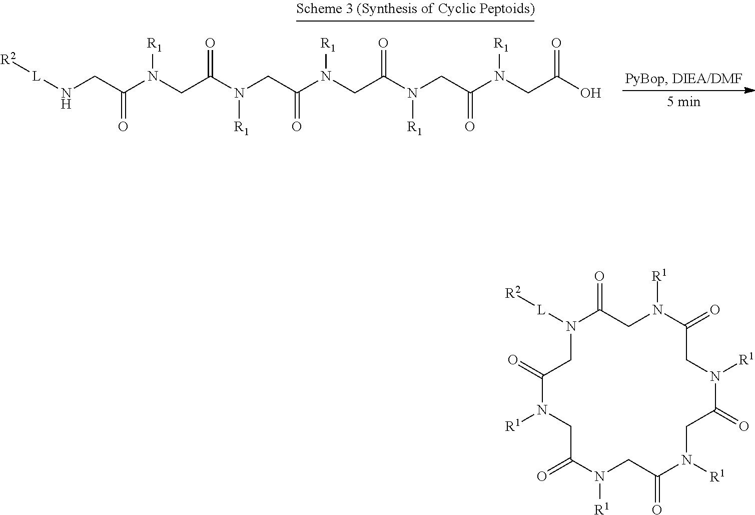

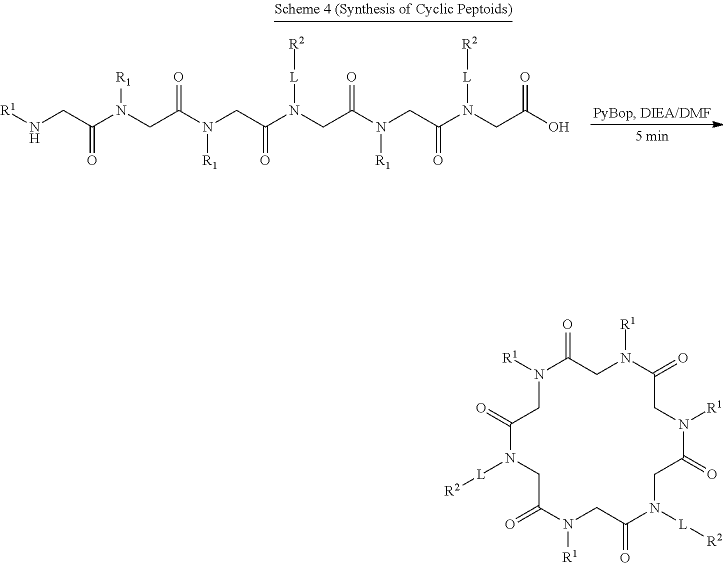

wherein L, R¹, and R² are as described herein and wherein:

| | |
|---|---|
| ACN | Acetonitrile |
| DCM: | Dichloromethane |
| DIC | Diisopropylcarbodiimide |
| DIEA | N,N-diisopropylethylamine |
| DMF | N,N-dimethylformamide |
| DMSO | Dimethylsulfoxide |
| HFIP | Hexafluoroisopropanol |
| NMR | Nuclear Magnetic Resonnance |
| PyBOP | (Benzotriazol-1-yloxy)tripyrrolidinophosphonium |
| | Hexafluorophosphate |
| TEA | Triethylamine |
| TEMPO | Tetramethylpiperidin-N-oxyl |
| TFA | Trifluoroacetic acid |

### EXAMPLES

The following examples are presented in order to more fully illustrate the preferred embodiments of the invention. They should in no way be construed, however, as limiting the broad scope of the invention.

### Representative Synthetic Methods

### Preparation of Peptoids of the Invention

### Example 1

### Preparation of Peptoid Oligomers

Peptoid oligomers were synthesized manually on Rink amide resin using the submonomer approach [Zuckermann, R. N.; Kerr J. M.; Kent S. B. W.; Moos W. H. J. Am. Chem. Soc. 1992, 114, 10646-10647]. All peptoid oligomers were synthesized at room temperature. Typically, 100 mg of resin was swollen in DCM for 40 minutes before starting oligomer synthesis. Multiple washing steps using DMF were performed between each step described below. Bromoacetylation was completed by adding 20 eq bromoacetic acid (1.2 M in DMF, 8.5 ml g⁻¹ resin) and 24 eq of diisopropylcarbodiimide (2 ml g⁻¹ resin); this reaction was allowed to shake at room temperature for 20 min. Following the reaction, the bromoacetylation reagents were washed from the resin using DMF (10 ml g⁻¹ resin) (3×1 min) and 20 equivalents of submonomer amine (1.0 M in DMF, 10 ml g⁻¹ resin) were added. The amine displacement reaction was allowed to shake at room temperature for 20 min and was followed by multiple washing steps (DMF, 10 ml g⁻¹ resin) (3×1 min).

Bromoacetylations and amine displacement steps were repeated until peptoid oligomers of desired sequence were obtained. To cleave the peptoid oligomers from solid support for analysis, approximately 5 mg of resin was treated with 95% TFA in water (40 ml g⁻¹ resin) for 10 minutes. The cleavage cocktail was evaporated under nitrogen gas and the peptoid oligomers were re-suspended in 0.5 ml HPLC solvent (1:1 HPLC grade acetonitrile:HPLC grade water). To cleave the peptoid oligomers from solid support for purification, 100 mg of resin was treated with 95% TFA in water (40 ml g⁻¹ resin) for 10 minutes. The cleavage cocktail was evaporated, re-suspended in 2 ml HPLC solvent, froze and lyophilized. In order to re-generate the TEMPO radical, the dry pink compound was dissolved in 9:1 ammonia 7N solution in methanol: water (4 ml for 100 mg resin) and stirred for 4 hours at 25° C. The solvent was then evaporated, re-suspended in 2 ml HPLC solvent, frozen and lyophilized. The dry compound was re-suspended in 0.5 mL HPLC solvent and injected to a preparative HPLC using a Delta-Pak C18 column (Waters, 15 μm, 100 Å, 25×100 mm). Peaks were eluted with a linear gradient of 5-95% ACN in water (0.1% TFA) over 50 min at a flow rate of 5 ml/min.

### Example 2

### i) Preparation of Cyclic Peptoid Oligomers

Peptoid oligomers were synthesized manually on 2-chlorotrityl chloride resin, using the submonomer approach [Zuckermann, R. N.; Kerr J. M.; Kent S. B. W.; Moos W. H. J. Am. Chem. Soc. 1992, 114, 10646-10647]. All peptoid oligomers were synthesized at room temperature. Typically, 200 mg of 2-chlorotrityl chloride resin was washed twice in 2 mL of DCM, followed by swelling in 2 mL of DCM. The first monomer was added by reacting 37 mg of bromoacetic acid (0.27 mmol; Sigma-Aldrich) and 189 μL of DIEA (1.08 mmol; Chem Impex International) in 2 mL of DCM on a shaker platform for 30 minutes at room temperature, followed by extensive washes with DCM (five times with 2 mL) and DMF (five times with 2 mL). Bromoacylated resin was incubated with 2 mL of 1M amine submonomer in DMF on a shaker platform for 30 minutes at room temperature, followed by extensive washes with DMF (five times with 2 mL). After that, all subsequent bromoacetylation and amine displacement steps were performed as follows: Bromoacetylation was completed by adding 20 eq bromoacetic acid (1.2 M in DMF, 8.5 ml g⁻¹ resin) and 24 eq of diisopropylcarbodiimide (2 ml g⁻¹ resin); this reaction was allowed to shake at room temperature for 20 min. Following the reaction, the bromoacetylation reagents were washed from the resin using DMF (10 ml g⁻¹ resin) (3×1 min) and 20 equivalents of submonomer amine (1.0 M in DMF, 10 ml g⁻¹ resin) were added. The amine displacement reaction was allowed to shake at room temperature for 20 min and was followed by multiple washing steps (DMF, 10 ml g⁻¹ resin) (3×1 min).

Bromoacetylations and amine displacement steps were repeated until peptoid oligomers of desired sequence were obtained. The peptoid-resin was cleaved in 2 mL of 20% HFIP (Alfa Aesar) in DCM (v/v) at room temperature. The cleavage was conducted in a glass tube with constant agitation for 30 minutes. HFIP/DCM was evaporated over stream of nitrogen gas. The final product was dissolved in 5 mL of 50% ACN in HPLC grade H₂O and filtered with a 0.5 μm stainless steel fritted syringe tip filter (Upchurch Scientific). Peptoid oligomers were analyzed on a C₁₈ reversed phase analytical HPLC column at room temperature (Peeke Scientific, 5 μm, 120 Å, 2.0×50 mm) using a Beckman Coulter System Gold instrument. A linear gradient of 5-95% acetonitrile/water (0.1% TFA, Acros Organics) over 20 min was used with a flow rate of 0.7 mL/min. Preparative HPLC was performed on a Delta-Pak C₁₈ (Waters, 15 μm, 100 Å, 25×100 mm) with a linear gradient of 5-95% acetonitrile/water (0.1% TFA) over 60 min with a flow rate of 5 mL/min. LC-MS was performed on an Agilent 1100 Series LC/MSD Trap XCT (Agilent Technologies). NMR data was collected with an Avance-400 NMR Spectrometer (Bruker).

### ii) General Cyclization Reaction

Typical cyclization reactions were conducted in dry, deoxygenated DMF. 12 pmoles of the linear peptoid was suspended in 5.25 mL of DMF in a 15 mL conical tube. 375 μL of PyBOP (NovaBiochem) solution (96 mM, freshly prepared in DMF) and 375 μL of DIEA (Chem Impex International) solution (192 mM, freshly prepared in DMF) were added to the peptoid. The reaction vessel was flushed with nitrogen and sealed to exclude air. The reaction proceeded for 5 minutes at room temperature and 10 μL of reaction mixture was diluted with 140 μL of 50% ACN in H₂O to quench the reaction. The diluted sample was analyzed using HPLC.

TABLE 1
PEPTOIDS OF THE INVENTION

| | | | Molecular |
|---|---|---|---|
| | Oligomer | Oligomer | weight |
| Peptoid | Sequence* | Length | Calc:Found |
| 1 | H-(Nspe)₃Ntempo(Nspe)₃-NH₂ | 7mer | 1196.5:1196.8 |
| 2 | H-(Nspe)₂Ntempo(Nspe)₄-NH₂ | 7mer | 1196.5:1196.8 |
| 3 | H-NspeNtempo(Nspe)₅-NH₂ | 7mer | 1196.5:1196.8 |
| 4 | H-Ntempo(Nspe)₆-NH₂ | 7mer | 1196.5:1196.8 |
| 4A | H-Ntempo(Nrpe)₆-NH₂ | 7mer | 1196.5:1196.2 |
| 5 | H-(Nspe)₂ NpmNtempoNspeNpmNspe-NH₂ | 7mer | 1168.5:1168.3 |
| 6 | H-Nspe(Npm)₂Ntempo(Npm)₂Nspe-NH₂ | 7mer | 1140.4:1140.3 |
| 6A | H-NspeNpmNspeNtempoNspeNpmNspe-NH₂ | 7mer | 1168.5:1168.3 |
| 7 | H-NtempoNspeNpm(Nspe)₂ NpmNspe-NH₂ | 7mer | 1168.5:1168.3 |
| 8 | H-Ntempo(Npm)₂Nspe(Npm)₂ Nspe-NH₂ | 7mer | 1140.4:1140.3 |
| 9 | H-Ntempo(Nspe)₅-NH₂ | 6mer | 1035.3:1035.5 |
| 10 | H-Ntempo(Nspe)₄-NH₂ | 5mer | 874.1:874.4 |
| 11 | H-Ntempo(Nspe)₃-NH₂ | 4mer | 712.9:713.4 |
| 12 | H-Ntempo(Nspe)₂-NH₂ | 3mer | 551.7:552.3 |
| 13 | H-(Npm)₃Ntempo(Npm)₃-NH₂ | 7mer | 1112.4:1112.5 |
| 14 | H-Ntempo(Npm)₆-NH₂ | 7mer | 1112.4:1112.5 |
| 15 | H-NtempoNrpeNpm(Nrpe)₂NpmNrpe-NH₂ | 7mer | 1168.7:1168.6 |
| 16 | H-Ntempo(Nspe)₃(Nrpe)₃-NH₂ | 7mer | 1196.7:1196.8 |
| 17 | H-Ntempo(Nsmp)₃NH₂ | 4mer | 616.8:617.4 |
| 18 | H-NtempoNsmpNme(Nsmp)₂NmeNsmp-NH₂ | 7mer | 976.2:976.7 |
| 19 | H-NspePropylazideNtempoNspePropagylNspe-NH₂ | 6mer | 948.2:948.2 |
| 20 | H-Propylazide(Nspe)₂NtempoNspePropagylNspe-NH₂ | 7mer | 1109.4:1109.3 |
| 21 | H-(Nspe)₄Ntempo(Nspe)₄-NH₂ | 9mer | 1518.9:1520.2 |
| 22 | H-Ntempo(Nspe)₃(Npm)₃-NH₂ | 7mer | 1154.4:1154.8 |
| 39 | Acetyl-Ntempo(Nspe)₆-NH₂ | 7mer | 1237.6:1238.8 |
| 40 | Cyclic(Ntempo(Nspe)₅) | 6mer | 1017.3:1018.2 |
| 41 | Cyclic(NtempoNpmNspeNpmNspeNpm) | 6mer | 975.2:976.2 |

*structures as depicted herein.

### Example 3

### Asymmetric Catalytic Resolution of Aromatic Secondary Alcohols Using TEMPO-Containing Peptoid as a Catalyst

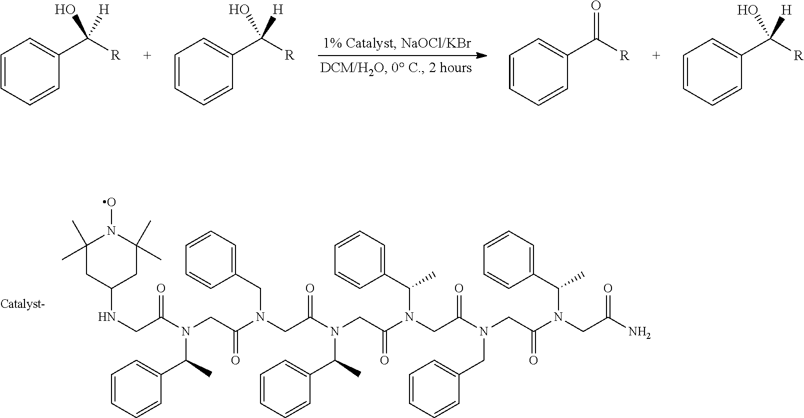

### Oxidation Catalysis: General Procedure

An 8 ml glass vial was charged with 1.2 mg peptoid (7mers, 1×10⁻⁴ mol), 0.25 ml CH₂Cl₂, 0.125 ml of 0.5M KBr in water and 1×10⁻⁴ mol substrate (alcohol), placed in an ice bath and cooled to 0° C. under stirring. The reaction started with the addition of 0.310 ml 0.5M NaOCl solution [1 equivalent of 1.8M NaOCl (that contains 10-13% Cl) and 2.6 equivalents of water]. After two hours, 1 ml CH₂Cl₂ was added, the aqueous layer was separated and a sample from the CH₂Cl₂ solution was analyzed by GC.

### REFERENCES

Jallabert C., Lapinze C. and Rivere H. J. Mol. Catal., 1980, 7, 127-136.
Marko I. E., Giles P. R., Brown S. M. and Urch C. J. Adv. Inorg. Chem., 2004, 56, 211-240.

TABLE 2
Bleach oxidation of Sec-phenethylalcohol using
TEMPO-containing peptoids off resin

| Peptoid sequence | Conversion, % | Selectivity, % | | ee, % | |
|---|---|---|---|---|---|
| H-(NsE)₃Ntempo (Nspe)₃-NH₂ | 56 | 52 | (R) | 5 | (S) |
| H-(Nspe)₃Ntempo (Nspe)₃-NH₂^{a} | 46 | 55 | (R) | 7 | (S) |
| H-NspeNpmNspe Ntempo NspeNpmNspe-NH₂ | 47 | 56 | (R) | 12 | (S) |
| H-NspeNpmNspe Ntempo NspeNpmNspe-NH₂^{a} | 45 | 58 | (R) | 12 | (S) |
| H-Nspe(Npm)₂ Ntempo (Npm)₂Nspe-NH₂ | 48 | 62 | (S) | 23 | (R) |
| H-Ntempo (Nspe)₆-NH₂ | 47 | 75 | (S) | 51 | (R) |
| H-Ntempo NspeNpm(Nspe)₂NpmNspe-NH₂ | 89 | 56 | (S) | >99 | (R) |
| H-Ntempo NrpeNpm(Nrpe)₂NpmNrpe-NH₂ | 88 | 56 | (R) | >99 | (S) |
| H-Ntempo (Npm)₂Nspe(Npm)₂Nspe-NH₂ | 44 | 57 | (S) | 11 | (R) |
| H-Ntempo (Nrpe)₃(Nspe)₃-NH₂ | 85 | 59 | (R) | >99 | (S) |
| H-Ntempo (Nspe)₆-NH₂ | 84 | 60 | (S) | >99 | (R) |
| H-Ntempo (Nrpe)₆-NH₂ | 85 | 59 | (S) | >99 | (S) |
| H-Ntempo(Nspe)₅-NH₂ | 63 | 35 | (S) | 60 | (S) |
| H-Ntempo(Nspe)₄-NH₂ | 77 | 22 | (S) | 53 | (S) |
| H-Ntempo(Nspe)₃-NH₂ | 71 | 22 | (S) | 55 | (S) |

Reaction conditions: Sec-phenethylalcohol 1 × 10⁻⁴ mol, P-TEMPO 1 × 10⁻⁶ mol (1:100), DCM 0.25 ml, KBr 0.5M 0.125 ml, NaOCl 0.5M 0.31 ml, 0° C., 2 hr. (a) on resin.

TABLE 3
Bleach oxidation of 1-phenyl-1-propanol using TEMPO-
containing peptoids off resin

| | Con- | Selec- | | | |
|---|---|---|---|---|---|
| | version, | tivity, | | | |
| Peptoid sequence | % | % | | ee, % | |
| H-(Nspe)₃ Ntempo (Nspe)₃-NH₂ | 74 | 51 | (R) | 8 | (S) |
| H-NspeNpmNspe Ntempo Npm(Nspe)₂- | 75 | 55 | (R) | 28 | (S) |
| NH₂ | | | | | |
| H-Nspe(Npm)₂ Ntempo (Npm)₂Nspe-NH₂ | 72 | 58 | (S) | 43 | (R) |
| H-Ntempo(Nspe)₆-NH₂ | 23 | 66 | (S) | 10 | (R) |
| H-Ntempo NspeNpm(Nspe)₂NpmNspe- | 92 | 54 | (S) | >99 | (R) |
| NH₂ | | | | | |
| H-Ntempo NrpeNpm(Nrpe)₂NpmNrpe- | 85 | 57 | (R) | >99 | (S) |
| NH₂ | | | | | |
| H-Ntempo (Npm)₂Nspe(Npm)₂Nspe-NH₂ | 56 | 56 | (S) | 18 | (R) |
| H-Ntempo (Npm)₃(Nspe)₃-NH₂ | 68 | 69 | (R) | 82 | (S) |

Reaction conditions: 1-phenyl-1-propanol 1 × 10⁻⁴ mol, Peptoid-TEMPO 1 × 10⁻⁶ mol (1:100), DCM 0.25 ml, KBr 0.5M 0.125 ml, NaOCl 0.5M 0.31 ml, 0° C., 2 hr.

### Example 4a

### Asymmetric Catalytic Resolution of Aromatic Secondary Alcohols Using Phenanthroline Containing Peptoid as a Catalyst

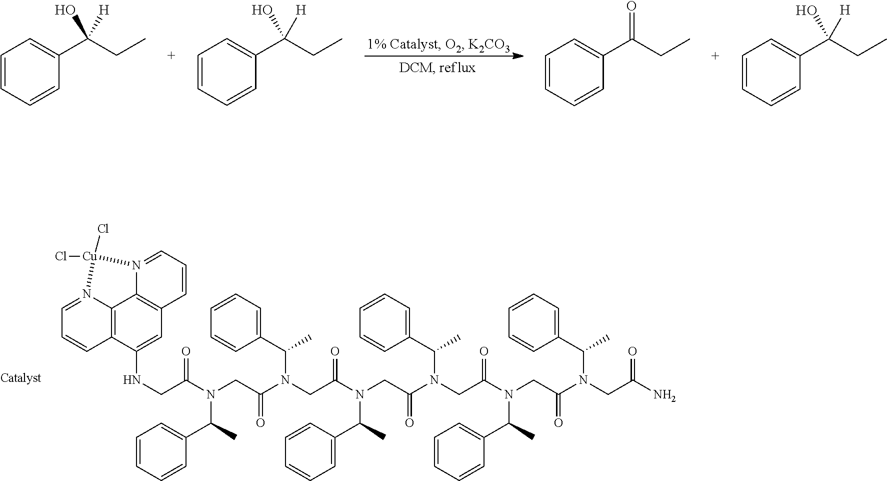

### Example 4b

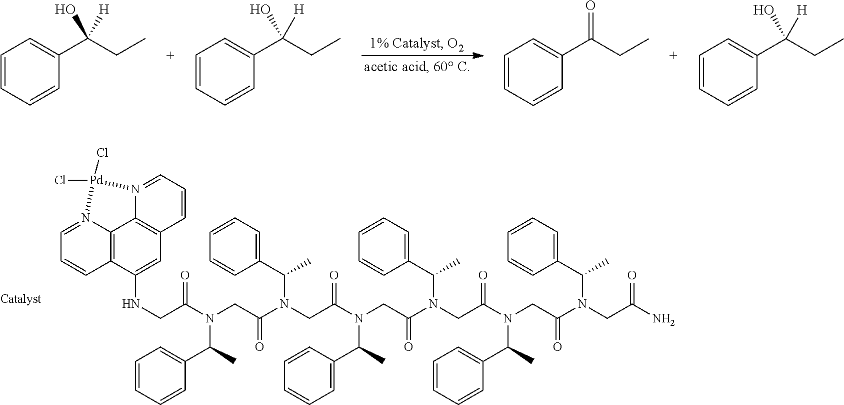

### Example 4c

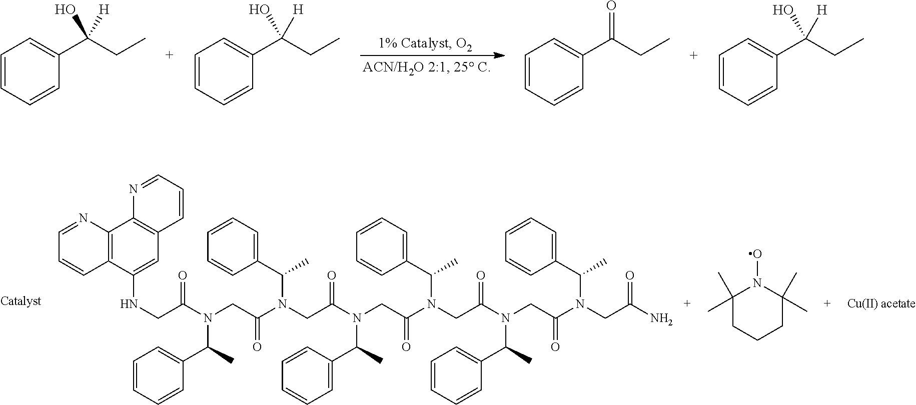

### Example 4d

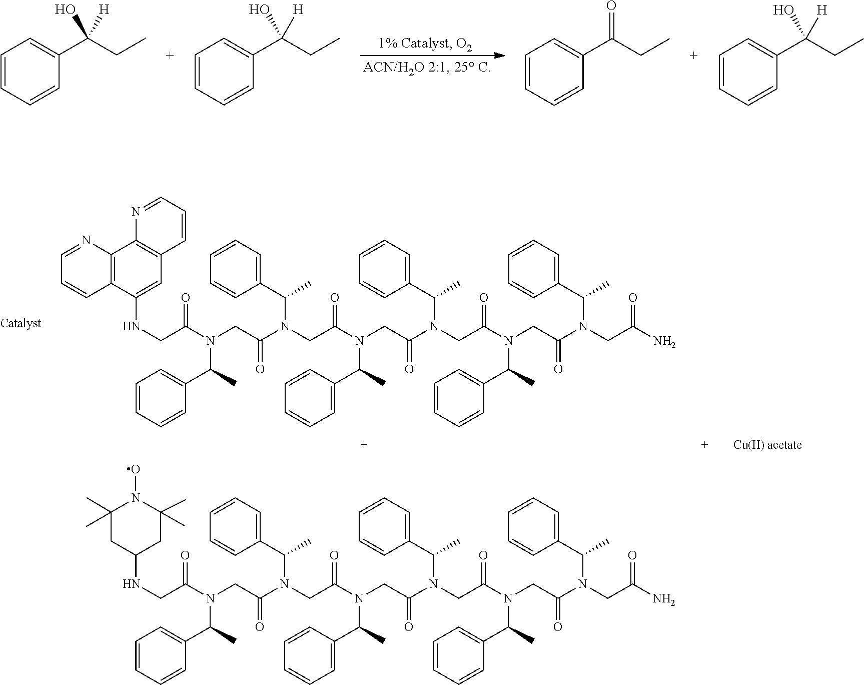

### Example 5

### Asymmetric Catalytic Resolution of Aromatic Secondary Alcohols

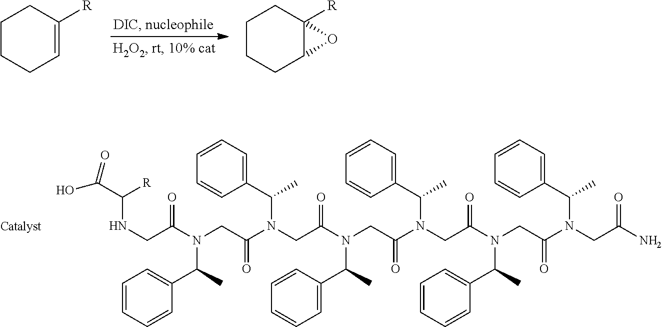

### Example 6A

### Regio-Selective Acylations of Polyols Using N-Methlylimidazole Containing Peptoid as a Catalyst

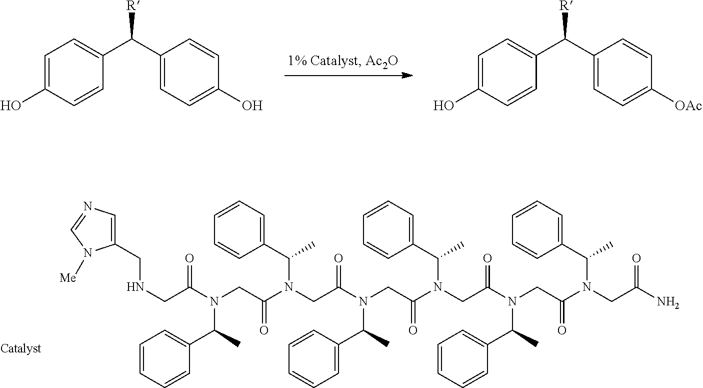

### Example 6B

### Regio-Selective Acylations of Polyols Using N-Methlylimidazole Containing Peptoid as a Catalyst

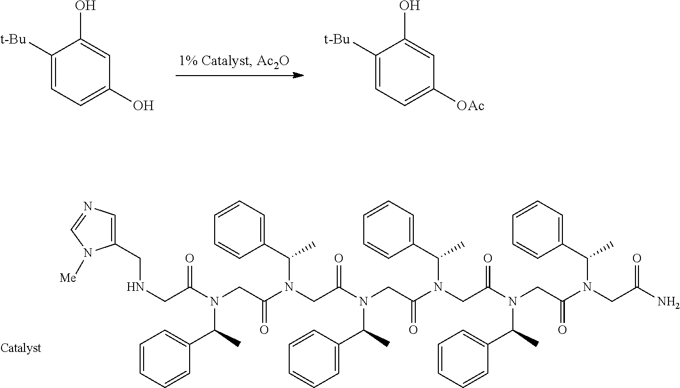 [SMILES: CC(C)(C)c1ccc(O)cc1O ; CC(=O)Oc1ccc(C(C)(C)C)c(O)c1 ; C[C@@H](c1ccccc1)N(CC(N)=O)C(=O)CN(C(=O)CN(C(=O)CN(C(=O)CN(C(=O)CN(C(=O)CNCc1cncn1C)[C@@H](C)c1ccccc1)[C@@H](C)c1ccccc1)[C@@H](C)c1ccccc1)[C@@H](C)c1ccccc1)[C@@H](C)c1ccccc1]

From the foregoing description, various modifications and changes in the compositions and methods of this invention will occur to those skilled in the art. All such modifications coming within the scope of the appended claims are intended to be included therein.

It is further understood that all base sizes or amino acid sizes, and all molecular weight or molecular mass values, given for nucleic acids or polycyclic peptoids are approximate, and are provided for description.

All publications, including but not limited to patents and patent applications, cited in this specification are herein incorporated by reference as if each individual publication were specifically and individually indicated to be incorporated by reference herein as though fully set forth.

1. A method for conducting a catalytic transformation selected from the group consisting of substrate-selective catalytic transformations; regio-selective catalytic transformations; asymmetric catalytic transformations; the synthesis of enantiomerically pure organic compounds; and asymmetric catalytic resolutions; or
a method for conducting a chemical reaction selected from hydrolysis, aldol reactions; aldol condensations; Diels-Alder reactions; electrochemical oxidations; Michael reactions;
epoxidation; hydrogenation; acylation; phosphorylation; region-selective and enantioselective nucleophilic transfer reactions; Baeyer-Villiger oxidation of carbonyl groups to esters; solution phase or heterogeneous catalytic transformations; and regio-selective acylation of polyols;
wherein said method comprises conducting said transformation with a catalyst, and said catalyst is a peptoid oligomer according to formula Ia or Ib: 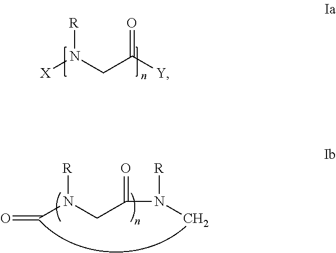
comprised of monomers according to formula II and formula III: 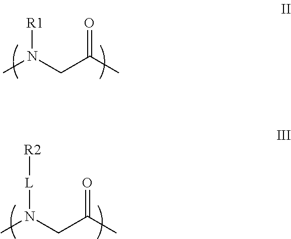
wherein
each R is independently substituted or unsubstituted alkyl, substituted or unsubstituted alkenyl, substituted or unsubstituted alkynyl, substituted or unsubstituted heterocycloalkyl, substituted or unsubstituted aryl, or substituted or unsubstituted heteroaryl;
each R¹ is independently substituted or unsubstituted alkyl, substituted or unsubstituted alkenyl, substituted or unsubstituted alkynyl, substituted or unsubstituted aryl or substituted or unsubstituted heteroaryl;
each R² is a conventional group capable of contributing to the catalysis of any organic transformation;
L is a single bond, C₁-C₄ alkylene, —C₂-C₄ alkylene-O—, or —C₂-C₄ alkylene-O—C₁-C₄ alkylene-;
X is H, substituted or unsubstituted acyl; Y is NH₂, OH, or acylamino, or acyloxy;
and n is an integer between 2-200;
or a salt thereof; and stereoisomers, isotopic variants and tautomers thereof.

2. The method according to claim 1, wherein 10-60% of the monomers are of formula III at the same time.

3. (canceled)

4. (canceled)

5. (canceled)

6. The method of claim 1, wherein R¹ is alkyl substituted with phenyl, naphthyl, alkoxy, or azido.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. The method of claim 1, wherein R¹ is

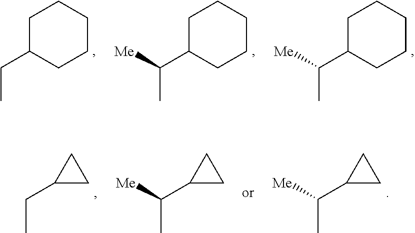

24. The method of claim 1, wherein R¹ is 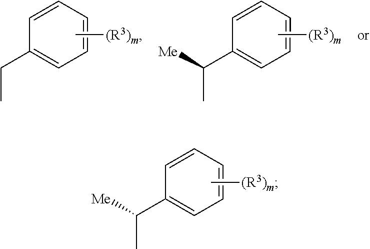
and wherein each R³ is independently alkyl, hydroxy, amino, nitro, or alkoxy and m is 0, 1 or 2.

25. (canceled)

26. The method of claim 1, wherein L is a single bond.

27. (canceled)

28. The method of claim 1, wherein L is —CH₂—CH₂—O—.

29. The method of claim 1, wherein R² is 8-hydroxyquinolinyl, phenanthrolinyl, terpyridinyl, amino, carboxy, sulfhydryl, imidazolyl, or phosphinyl, or metal complexes thereof.

30. The method of claim 1, wherein R² is 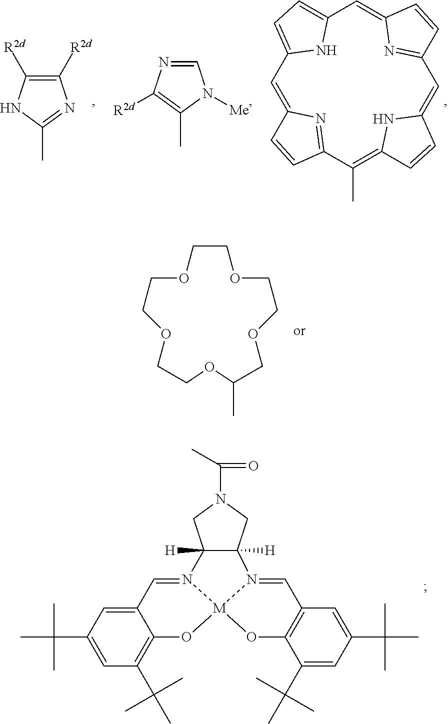
M is Ag, Au, Co, Cu, Fe, Mn, Ni, Pd, Pt, Rh, Ru, or Zn; and R^{2d} is halo, alkyl, or aryl.

31. The method of claim 1, wherein R² is —SH, or —CH(Me)NH₂.

32. The method of claim 1, wherein R² is a nitroxide containing group.

33. The method of claim 1, wherein R² is 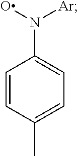
wherein Ar is aryl.

34. The method of claim 1, wherein R² is nitroxide containing heterocycloalkyl, or nitroxide containing heteroaryl.

35. The method of claim 1, wherein R² is

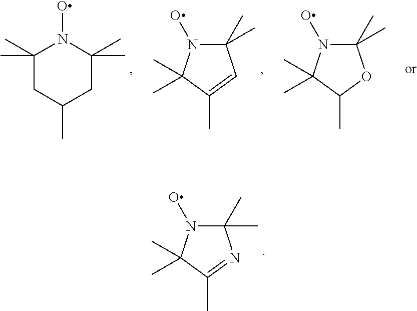

36. (canceled)

37. (canceled)

38. The method of claim 1, wherein the peptoid is of formula Ia or Ib; X, Y, R, R, R², L and n are as in claim 1; and each monomer of formula II is independently selected from Npm, Nme, Nspm, Naz, Nyl, Nspe, Nrpe, Nsch, and Nrch; and wherein

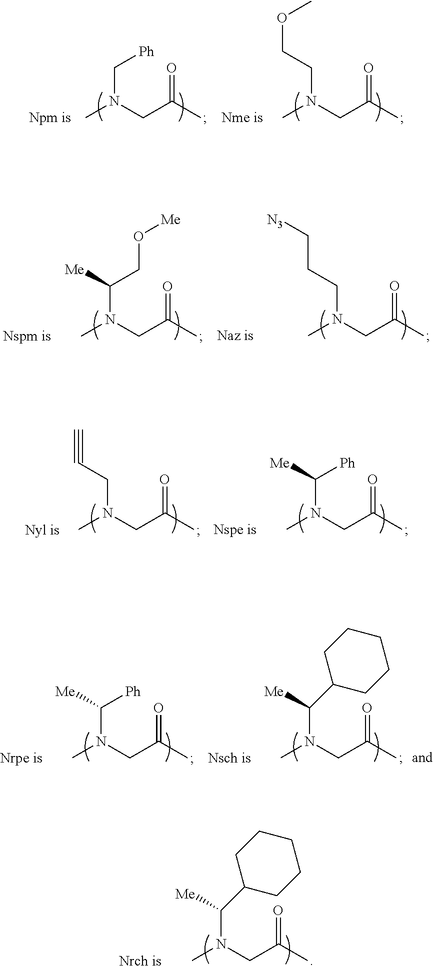

39. The method of claim 1, wherein n is 3-20.

40. (canceled)

41. (canceled)

42. The method of claim 1, wherein X is H or Ac.

43. (canceled)

44. The method of claim 1, wherein Y is OH, OAc, NH₂ or NHAc.

45. (canceled)

46. (canceled)

47. The method of claim 38, wherein n is 2-11; one monomer is of formula III; and the other monomers are independently selected from Npm, Nme, Nspm, Naz, Nyl, Nspe, Nrpe, Nsch, and Nrch.

48. (canceled)

49. The method of claim 38, wherein n is 3, 4, 5, 6, 7, or 9; and the peptoid is selected from the group consisting of H—N(L-R²)CH₂C(O)—(Nspe)₆-NH₂; H-(Nspe)₃-N(L-R²)CH₂C(O)—(Nspe)₃-NH₂; H-(Nspe)-(Npm)-Nspe-N(L-R²)CH₂C(O)—Nspe-Npm-Nspe-NH₂; H-(Nspe)-(Npm)₂-N(L-R²)CH₂C(O)—(Npm)₂-Nspe-NH₂; H—N(L-R²)CH₂C(O)—(Nspe)₆-NH₂; H—N(L-R²)CH₂C(O)—Nspe-Npm-(Nspe)₂-Npm-Nspe-NH₂; H—N(L-R²)CH₂C(O)—Nrpe-Npm-(Nrpe)₂-Npm-Nrpe-NH₂; H—N(L-R²)CH₂C(O)—(Npm)₂-Nspe-(Npm)₂-Nspe-NH₂; H-(Npm)₃-N(L-R²)CH₂C(O)— (Npm)₃-NH₂; H—N(L-R²)CH₂C(O)— (Npm)₆-NH₂; H—N(L-R²)CH₂C(O)—NrpeNpm(Nrpe)₂NpmNrpe-NH₂; H—N(L-R²)CH₂C(O)— (Nspe)₃(Nrpe)₃-NH₂; H—N(L-R²)CH₂C(O)—NsmpNme(Nsmp)₂NmeNsmp-NH₂; H-Naz(Nspe)₂-N(L-R²)CH₂C(O)—NspeNylNspe-NH₂; H—N(L-R²)CH₂C(O)—(Nspe)₃(Npm)₃-NH₂; H—N(L-R²)CH₂C(O)— (Nspe)₅-NH₂; H-NspeNaz-N(L-R²)CH₂C(O)—NspeNylNspe-NH₂; H—N(L-R²)CH₂C(O)—(Nspe)₄-NH₂; H—N(L-R²)CH₂C(O)— (Nspe)₃-NH₂; H—N(L-R²)CH₂C(O)— (Nsmp)₃-NH₂; H—N(L-R²)CH₂C(O)—(Nspe)₂-NH₂; and H-(Nspe)₄-N(L-R²)CH₂C(O)—(Nspe)₄-NH₂.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. The method of claim 38, wherein the peptoid is selected from the group consisting of:

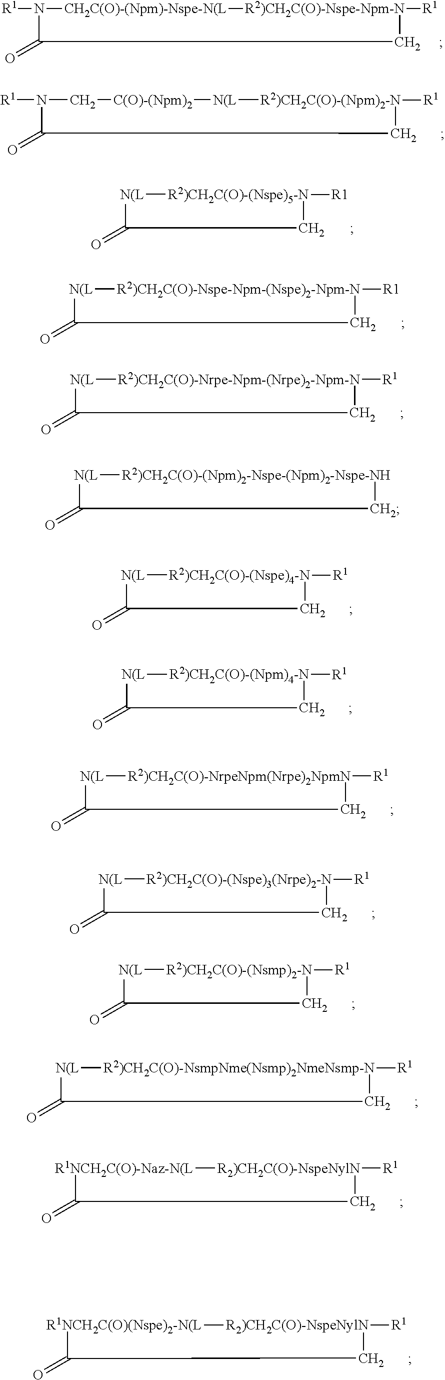

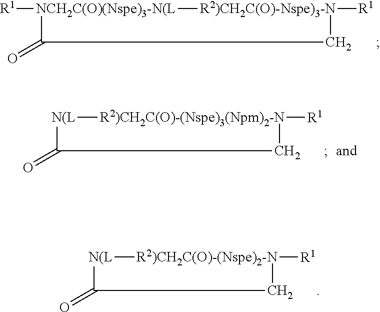

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. The method of either of claims 1 or 71, wherein R¹ is

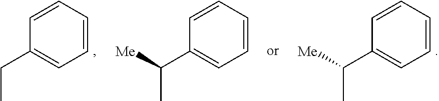

89. The method of either of claims 1 or 71, wherein R¹ is

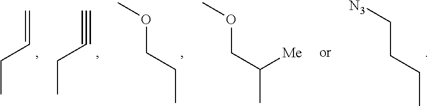

90. The method of claim 49, wherein L is a single bond; and L-R² is

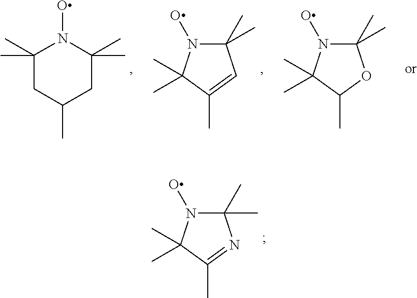

91. The method of claim 49, wherein L is a single bond; L-R² is 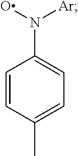
wherein Ar is aryl.

92. The method of claim 49, wherein -L-R² is 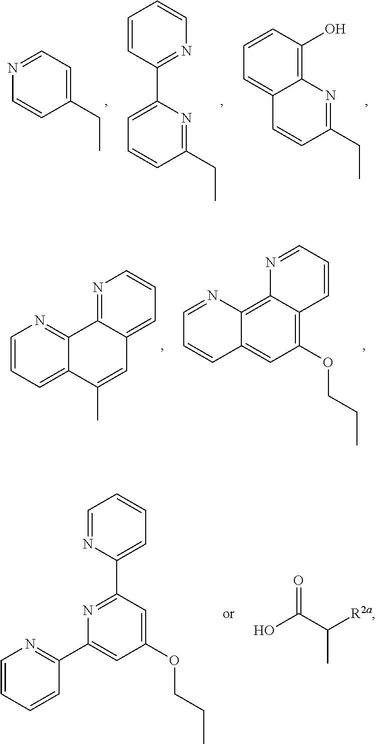
and wherein R^{2a} is substituted or unsubstituted alkyl or aryl.

93. The method of claim 49, wherein -L-R² is 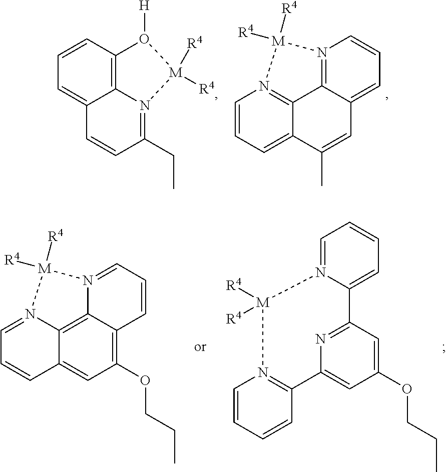
wherein M is Ag, Au, Co, Cu, Fe, Mn, Ni, Pd, Pt, Rh, Ru, or Zn; and R⁴ is Cl, Br, I, alkyl, aryl, hydroxy, SH, SO₃H, SO₂-aryl, or SO₂-alkyl.

94. The method of claim 49, wherein -L-R² is 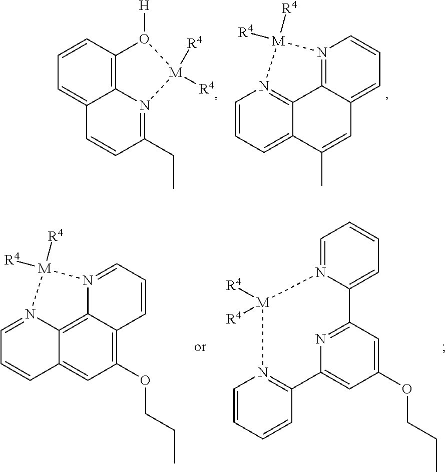
wherein M is Ag, Au, Co, Cu, Fe, Mn, Ni, Pd, Pt, Rh, Ru, or Zn; and R is Cl.

95. The method of claim 1, wherein -L-R² is

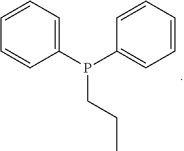 [SMILES: CCCP(c1ccccc1)c1ccccc1]

96. The method of claim 1, wherein -L-R² is

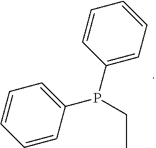

97. The method of claim 1, wherein the peptoid is selected from:
X-(Nspe)₃Ntempo(Nspe)₃-Y
X-(Nspe)₂Ntempo(Nspe)₄-Y
X-NspeNtempo(Nspe)₅-Y
X-Ntempo(Nspe)₆-Y
X-Ntempo(Nrpe)₆-Y
X-(Nspe)₂ NpmNtempoNspeNpmNspe-Y
X-Nspe(Npm)₂Ntempo(Npm)₂ Nspe-Y
X-NtempoNspeNpm(Nspe)₂ NpmNspe-Y
X-Ntempo(Npm)₂ Nspe(Npm)₂ Nspe-Y
X-Ntempo(Nspe)₅-Y
X-Ntempo(Nspe)₄-Y
X-Ntempo(Nspe)₃-Y
X-Ntempo(Nspe)₂-Y
X-(Npm)₃Ntempo(Npm)₃-Y
X-Ntempo(Npm)₆-Y
X-NtempoNrpeNpm(Nrpe)₂NpmNrpe-Y
X-Ntempo(Nspe)₃(Nrpe)₃-Y
X-Ntempo(Nsmp)₃-Y
X-NtempoNsmpNme(Nsmp)₂NmeNsmp-Y
X-NspePropylazideNtempoNspePropagylNspe-Y
X-Propylazide(Nspe)₂NtempoNspePropagylNspe-Y
X-(Nspe)₄Ntempo(Nspe)₄-Y
X-Ntempo(Nspe)₃(Npm)₃-Y and
X-NspeNpmNspeNtempoNspeNpmNspe-Y
and wherein X, and Y are as in claim 1;

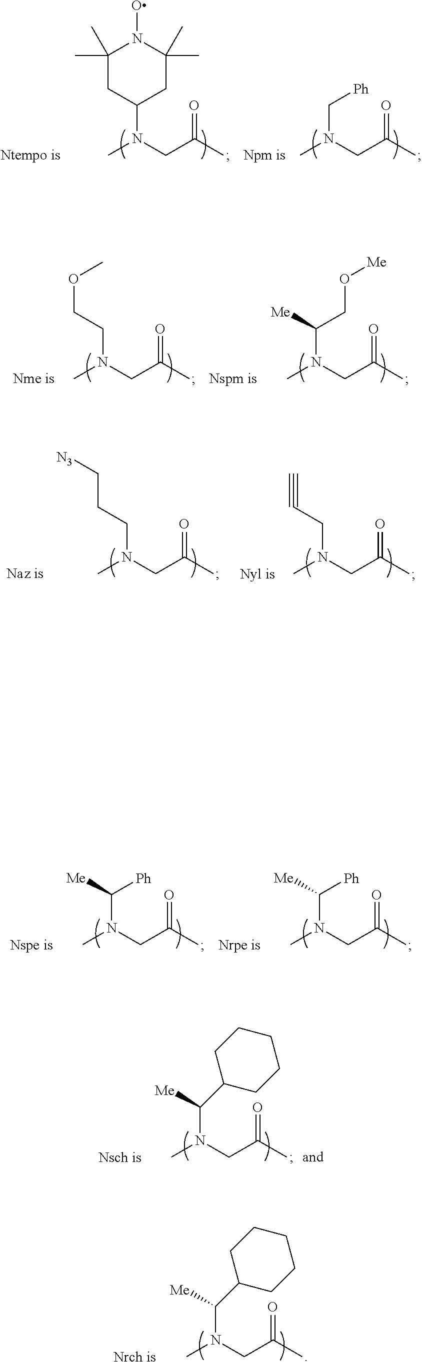

98. The method of claim 97, wherein X is H or Ac.

99. (canceled)

100. The method of claim 97, wherein Y is OH, OAc, NH₂ or NHAc.

101. (canceled)

102. (canceled)

103. The method of claim 1, wherein the peptoid is any one of peptoid selected from peptoids 1-27, 27A, 27B, 28-41, and 42:

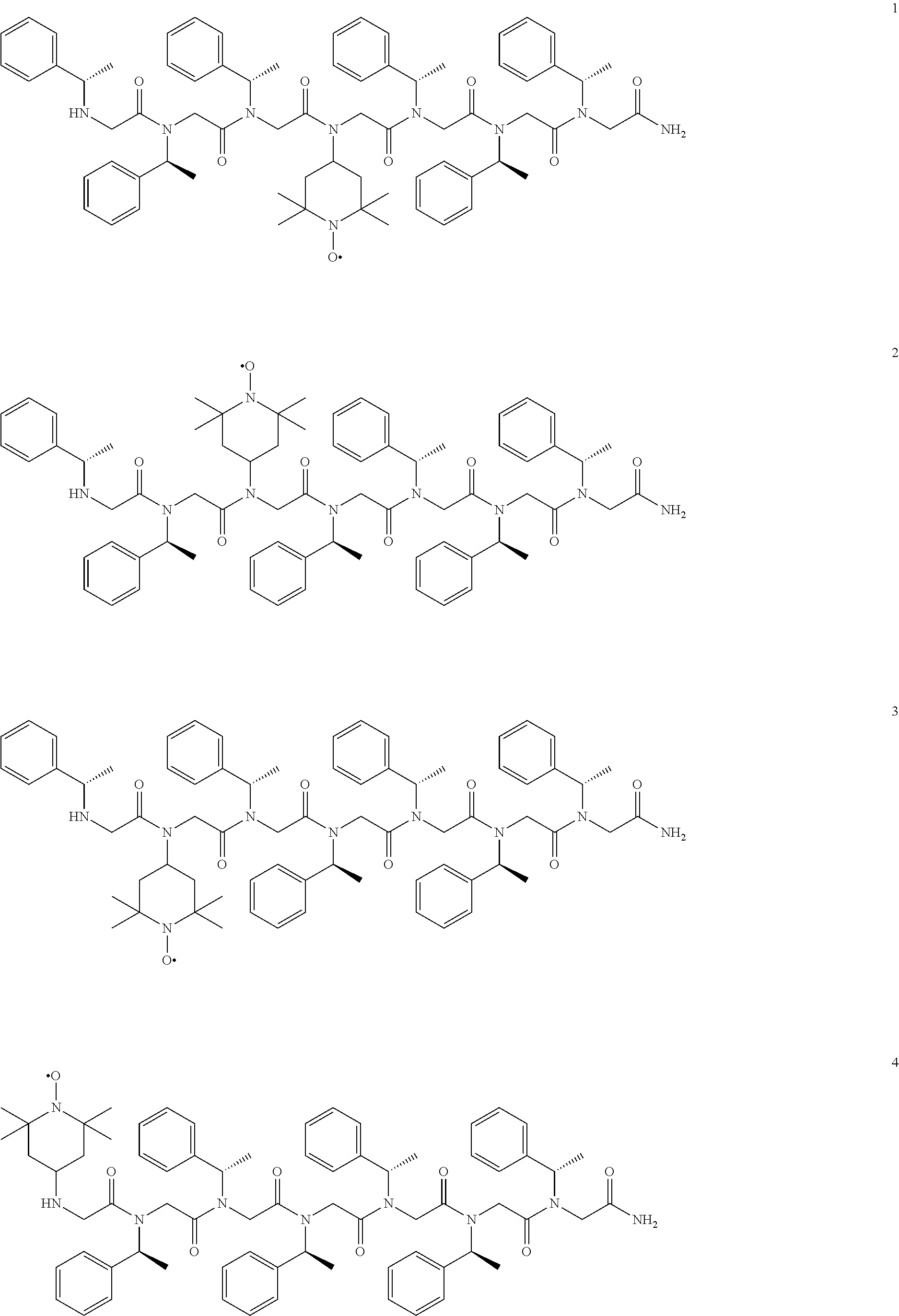

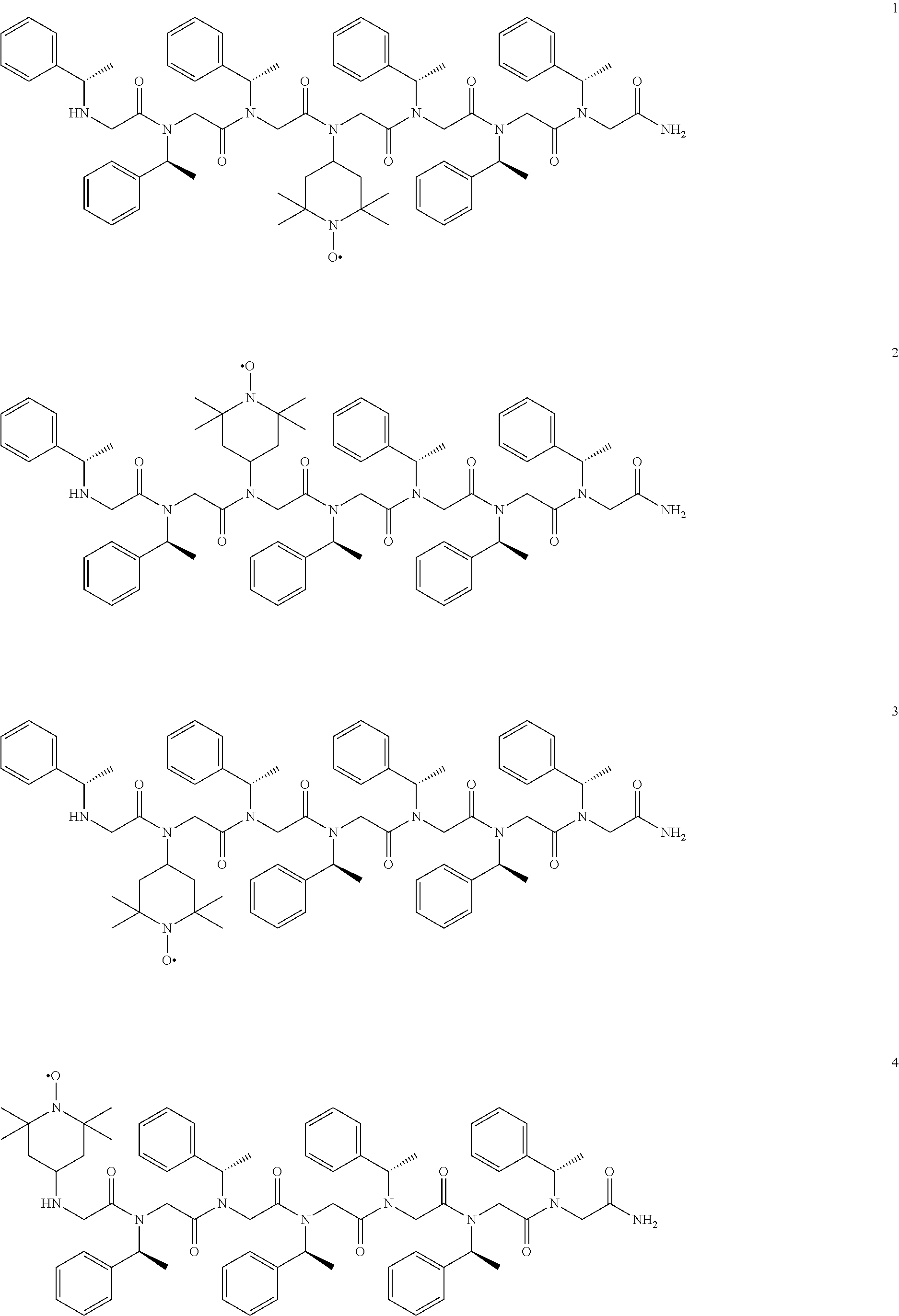

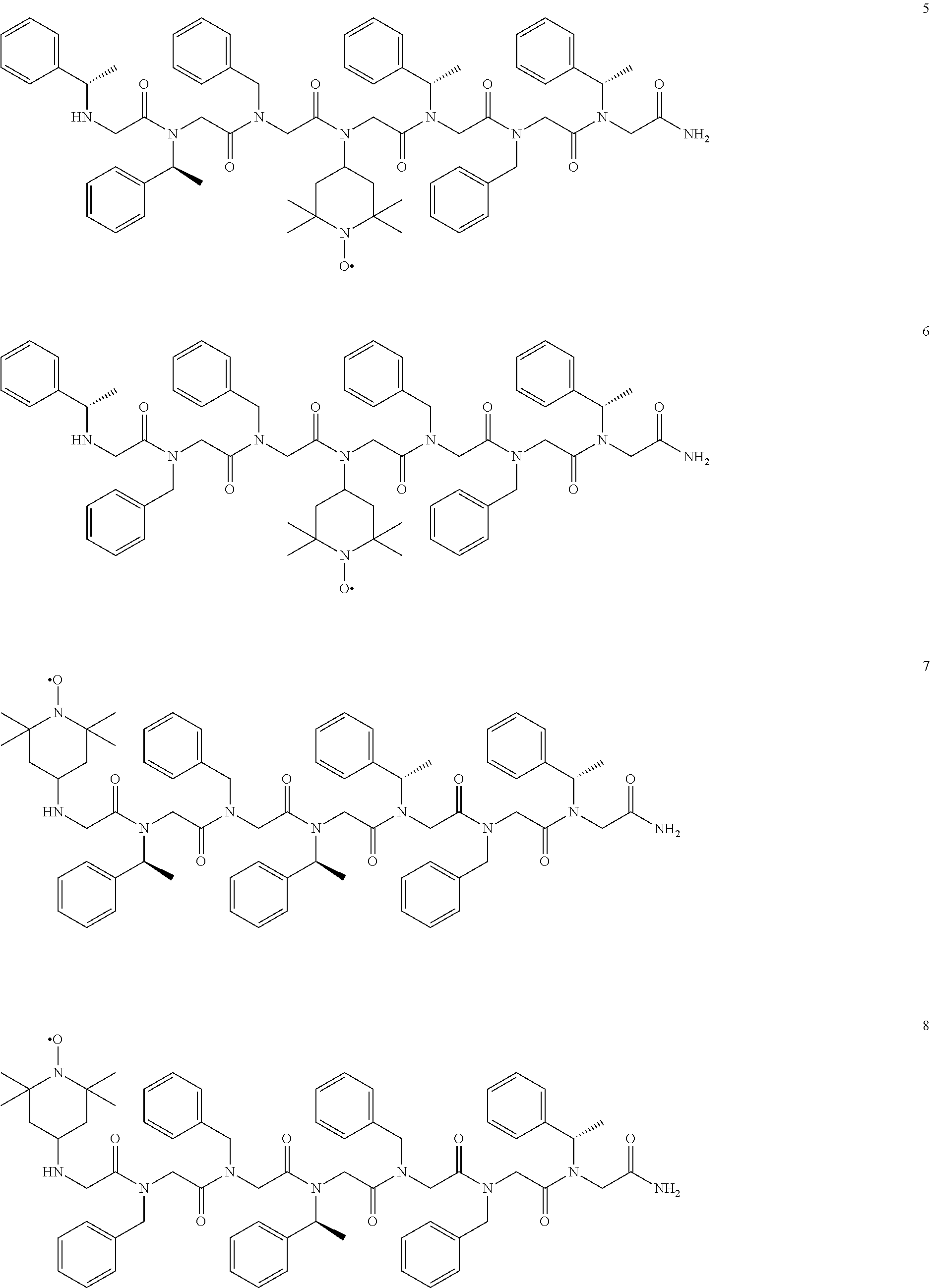

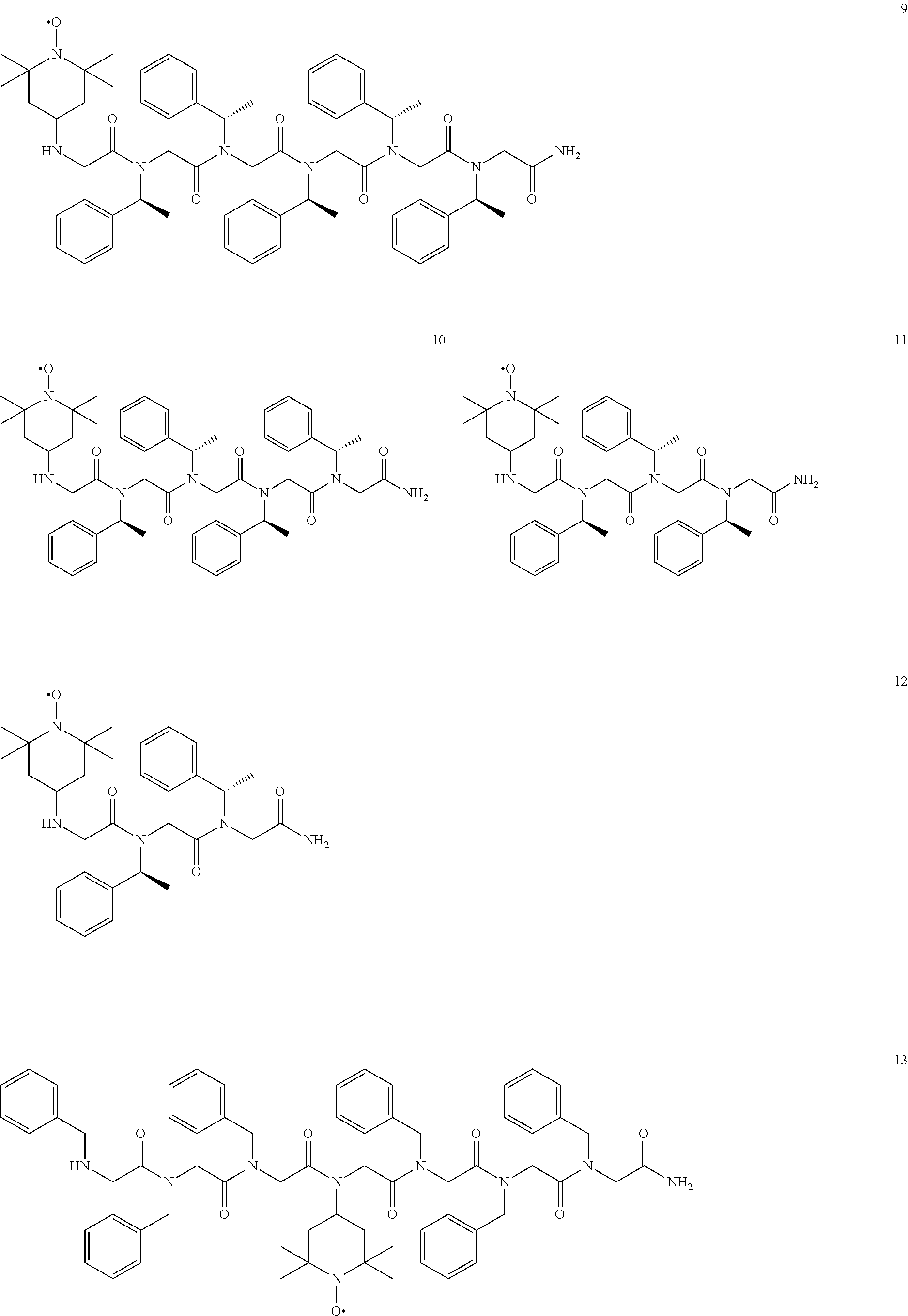

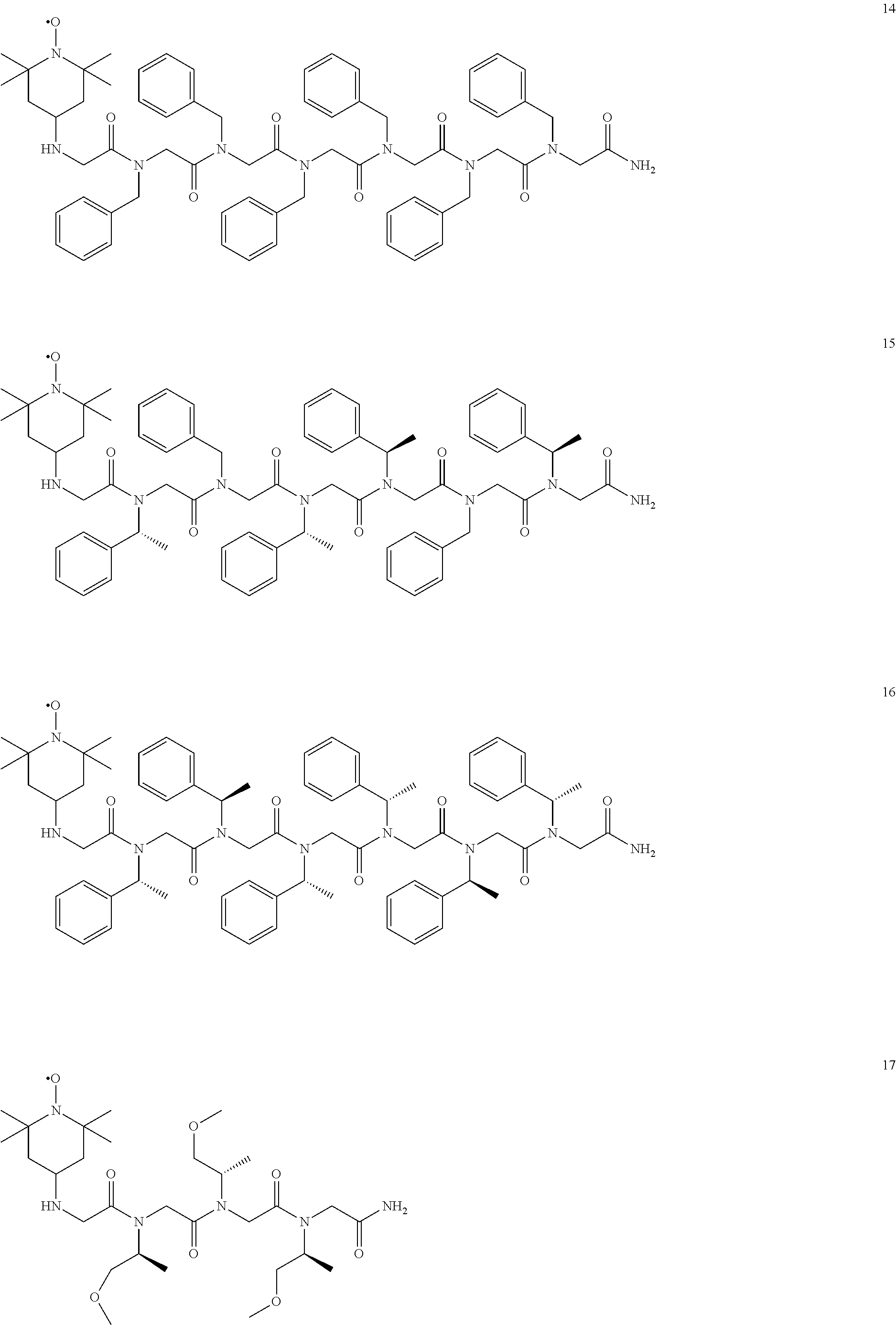

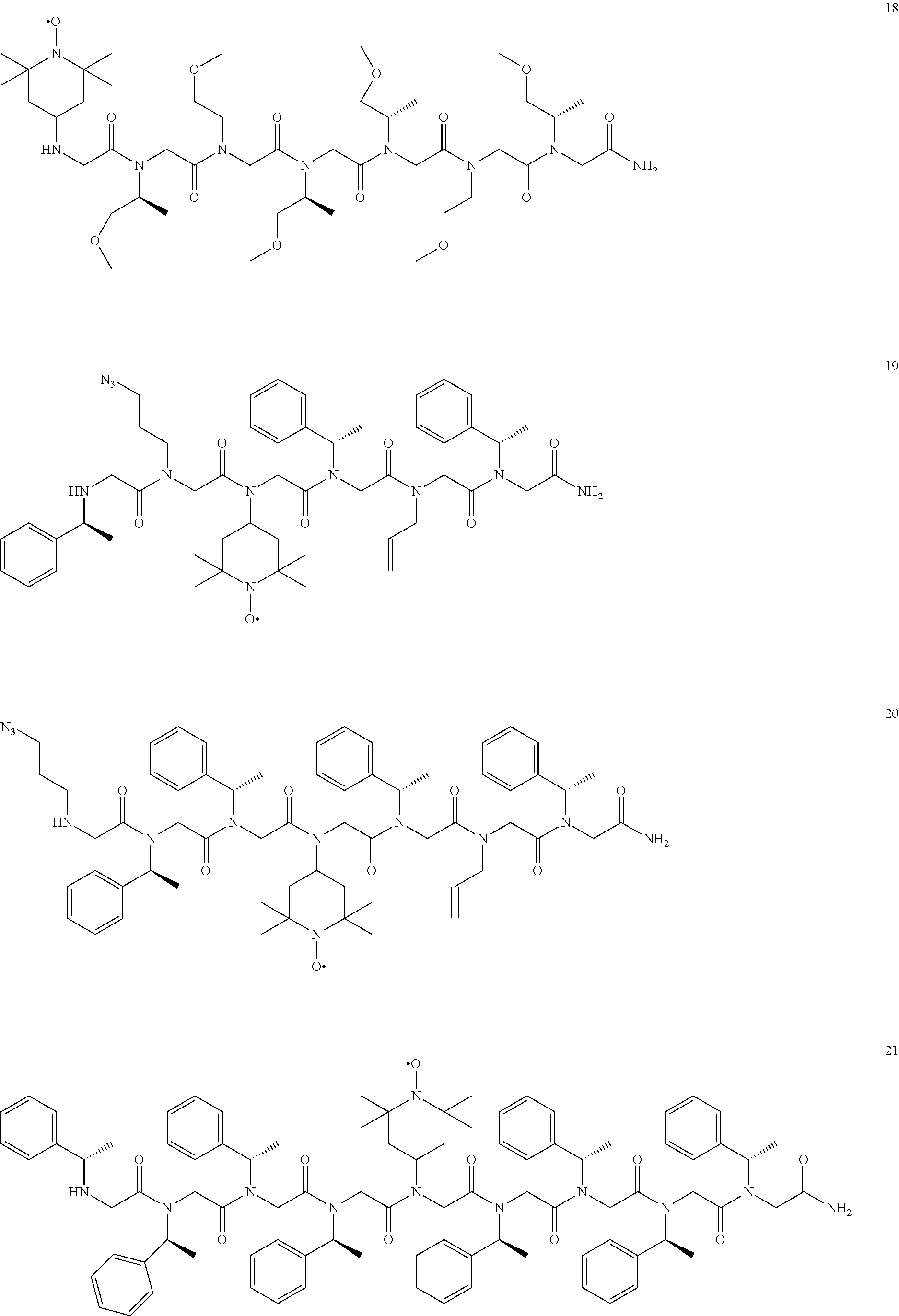

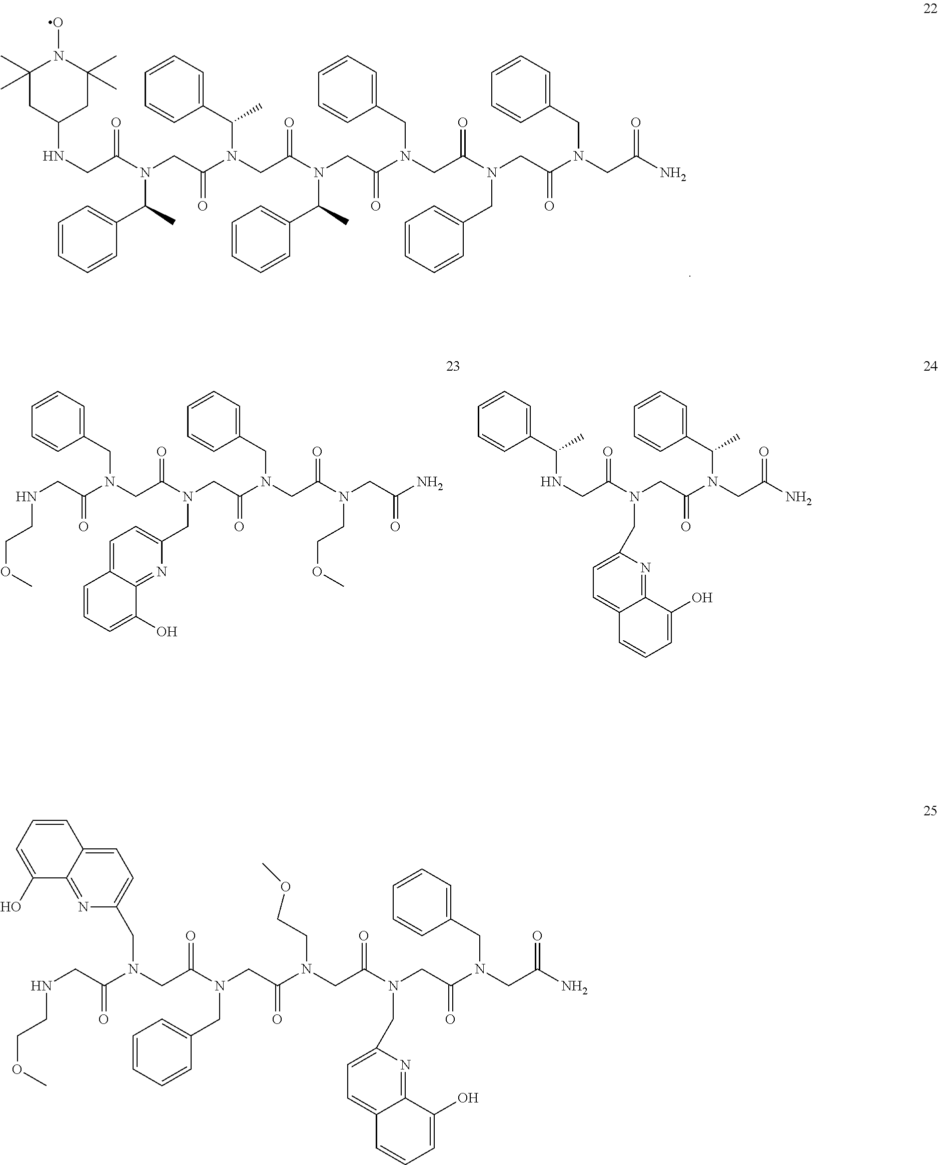

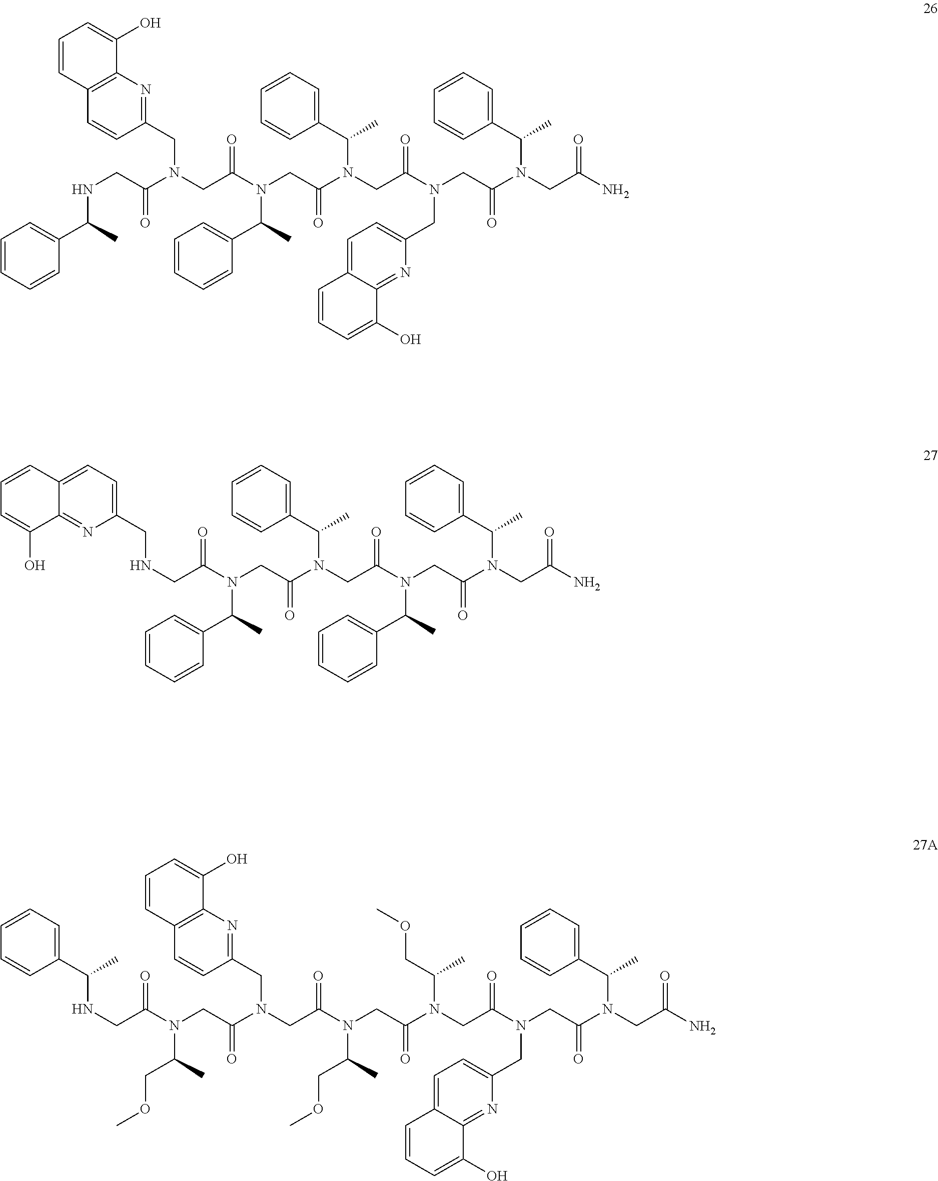 [SMILES: C[C@H](NCC(=O)N(CC(=O)N(CC(=O)N(CC(=O)N(CC(=O)N(CC(N)=O)[C@@H](C)c1ccccc1)Cc1ccc2cccc(O)c2n1)[C@@H](C)c1ccccc1)[C@@H](C)c1ccccc1)Cc1ccc2cccc(O)c2n1)c1ccccc1 ; C[C@@H](c1ccccc1)N(CC(N)=O)C(=O)CN(C(=O)CN(C(=O)CN(C(=O)CNCc1ccc2cccc(O)c2n1)[C@@H](C)c1ccccc1)[C@@H](C)c1ccccc1)[C@@H](C)c1ccccc1 ; COC[C@H](C)N(CC(=O)N(CC(=O)N(CC(=O)N(CC(=O)N(CC(=O)N(CC(N)=O)[C@@H](C)c1ccccc1)Cc1ccc2cccc(O)c2n1)[C@@H](C)COC)[C@@H](C)COC)Cc1ccc2cccc(O)c2n1)C(=O)CN[C@@H](C)c1ccccc1]

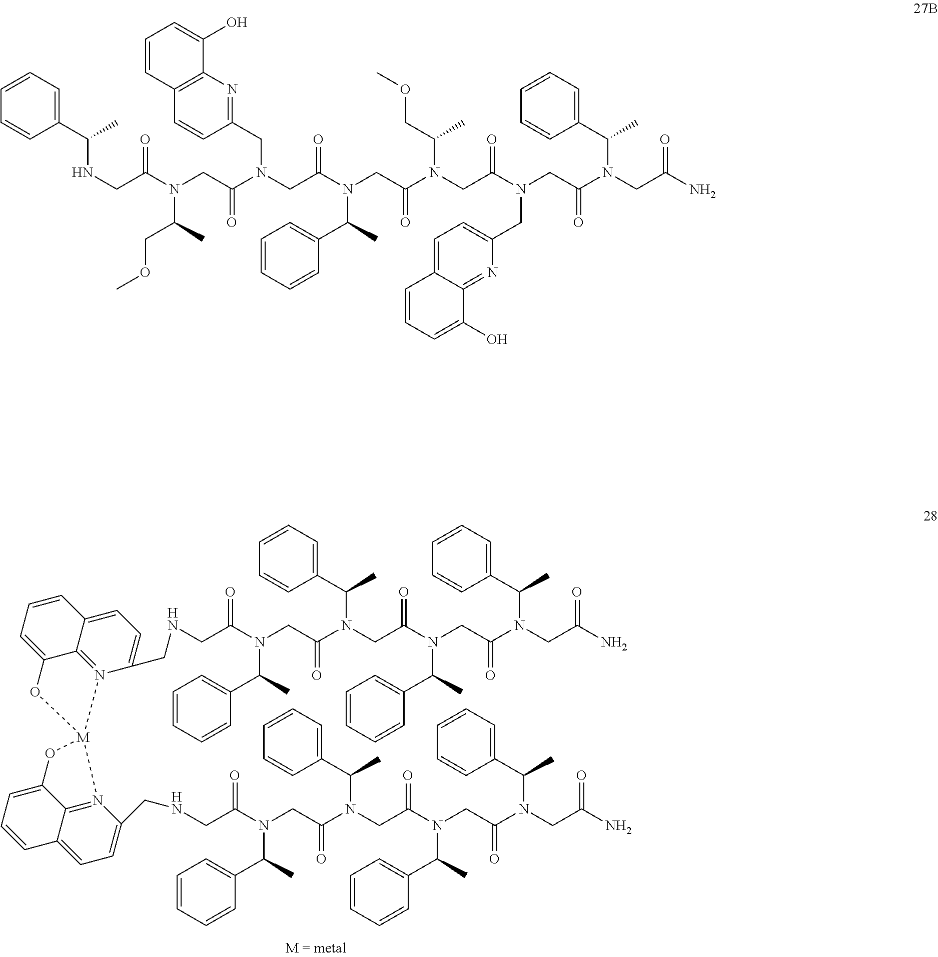

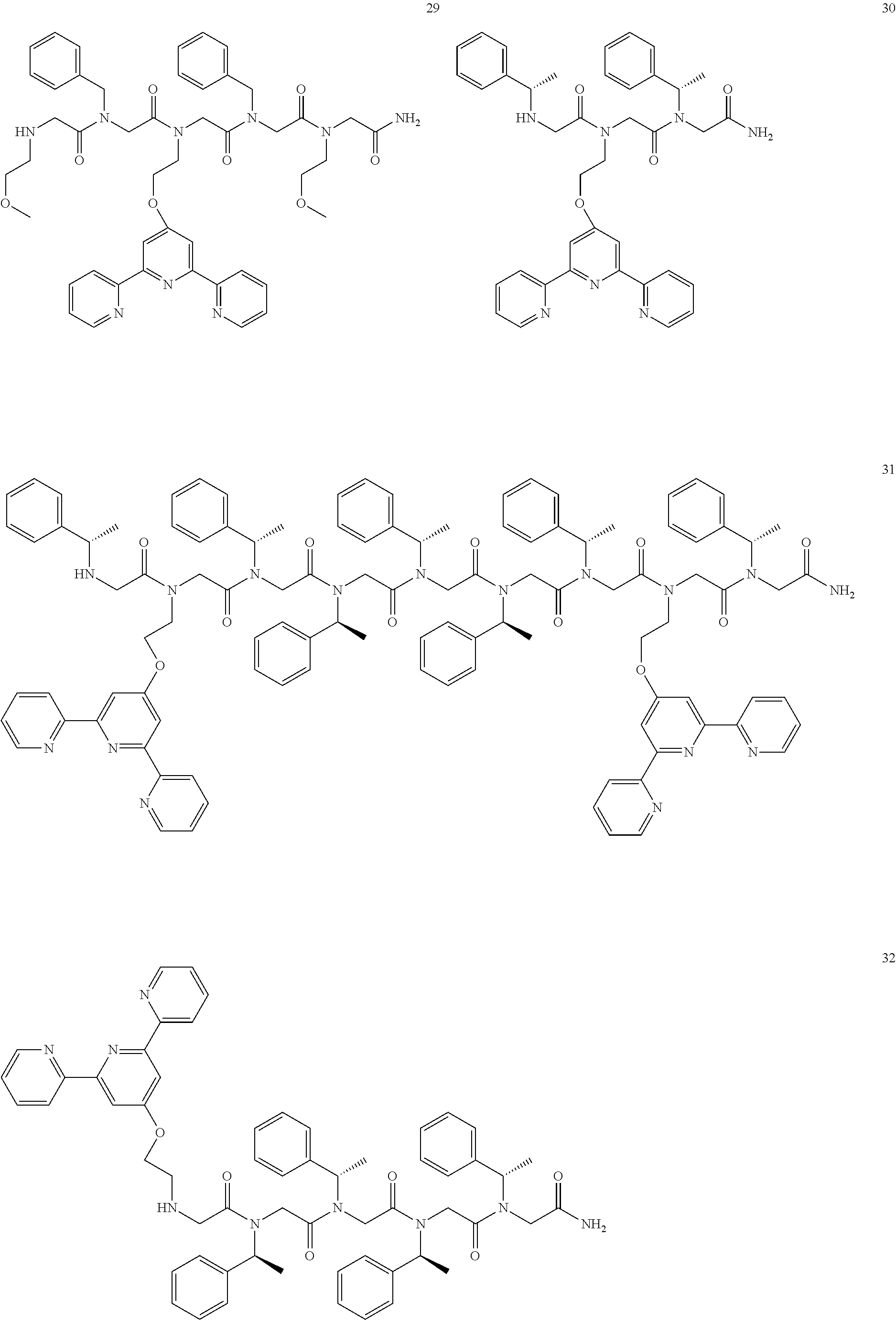

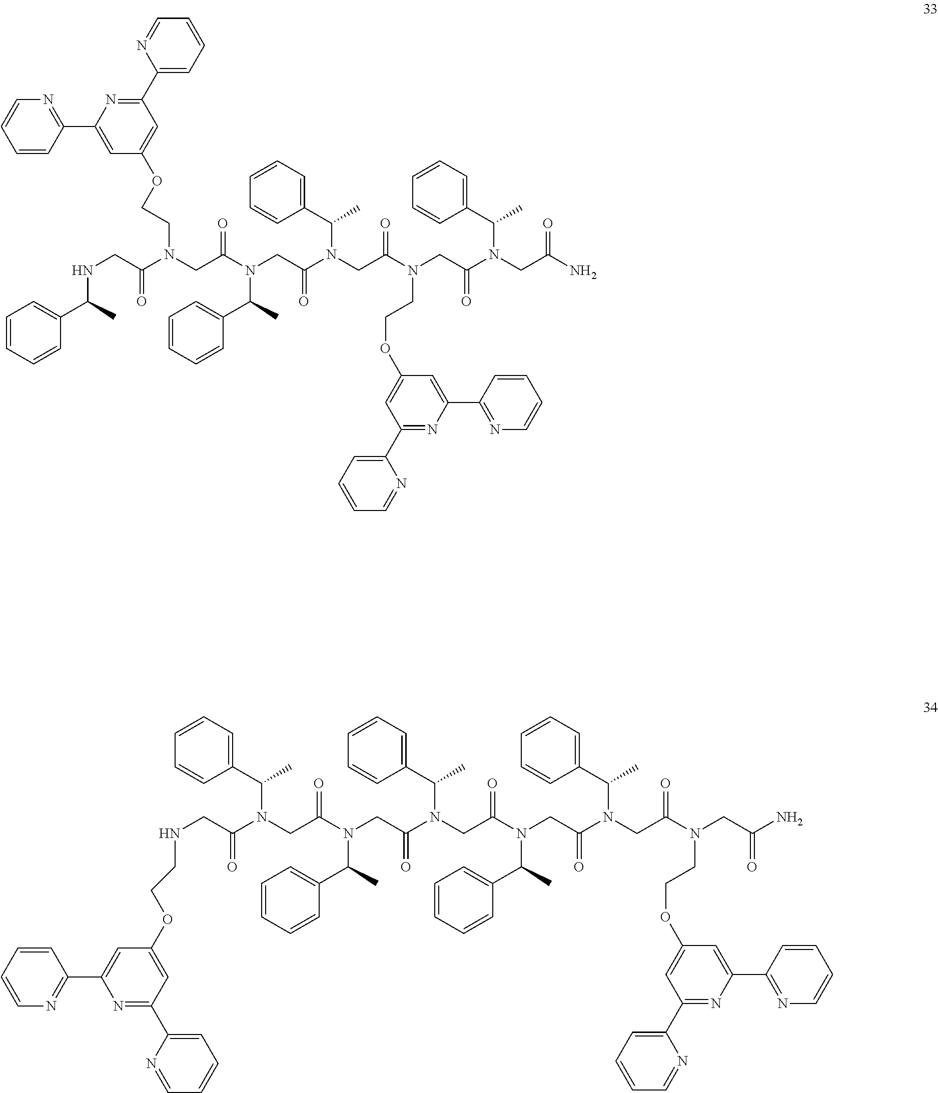 [SMILES: C[C@H](NCC(=O)N(CCOc1cc(-c2ccccn2)nc(-c2ccccn2)c1)CC(=O)N(CC(=O)N(CC(=O)N(CCOc1cc(-c2ccccn2)nc(-c2ccccn2)c1)CC(=O)N(CC(N)=O)[C@@H](C)c1ccccc1)[C@@H](C)c1ccccc1)[C@@H](C)c1ccccc1)c1ccccc1 ; C[C@@H](c1ccccc1)N(CC(=O)N(CCOc1cc(-c2ccccn2)nc(-c2ccccn2)c1)CC(N)=O)C(=O)CN(C(=O)CN(C(=O)CN(C(=O)CN(C(=O)CNCCOc1cc(-c2ccccn2)nc(-c2ccccn2)c1)[C@@H](C)c1ccccc1)[C@@H](C)c1ccccc1)[C@@H](C)c1ccccc1)[C@@H](C)c1ccccc1]

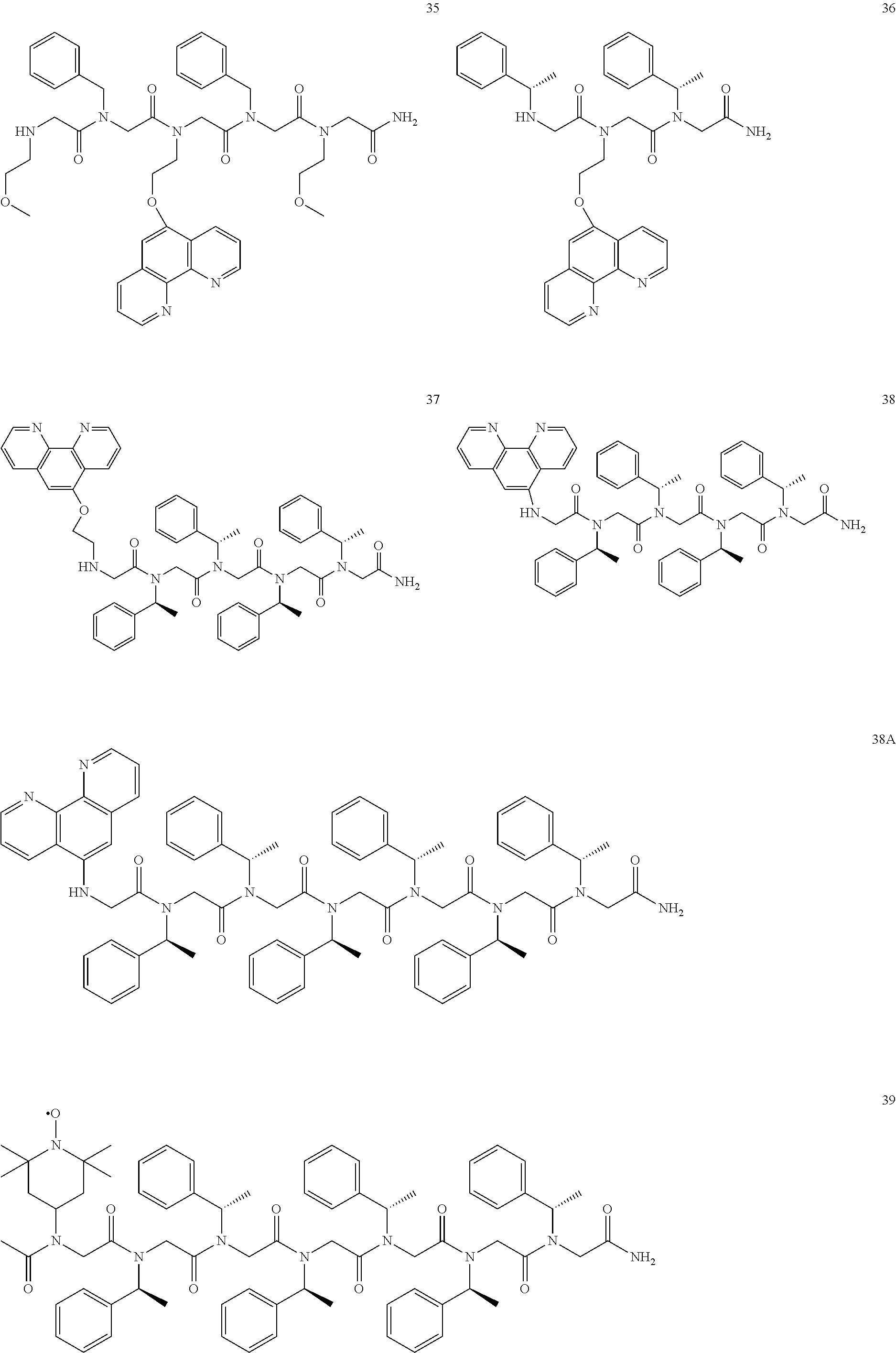

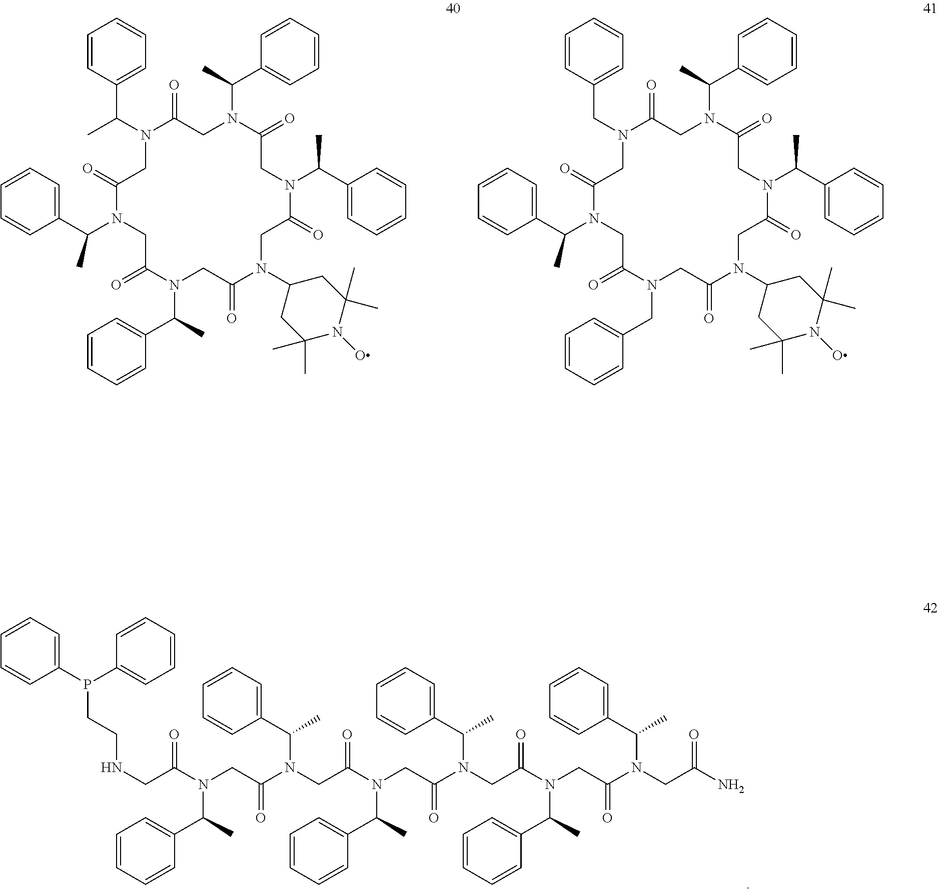

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. The method of claim 1, wherein the method is for conducting a catalytic transformation; and wherein the catalytic transformation is selected from substrate-selective catalytic transformations, regio-selective catalytic transformation, asymmetric catalytic transformations the synthesis of enantiomerically pure organic compounds; and asymmetric catalytic resolution.

118. The method of claim 1, wherein the method is for conducting a chemical reaction; wherein the chemical reaction is selected from hydrolysis, aldol reaction, aldol condensation, Diels-Alder reaction, electrochemical oxidation, Michael reaction, epoxidation, hydrogenation; acylation; phosphorylation; regio-selective and enantioselective nucleophilic transfer reaction, Baeyer-Villiger oxidation of carbonyl groups to esters; solution phase or heterogeneous catalytic transformation-s, and regio-selective acylation of polyols.

119. (canceled)

120. (canceled)
